(19) United States
(12) Patent Application Publication
(10) Pub. No.: US 2012/0263740 A1
(43) Pub. Date: Oct. 18, 2012

(54) APTAMER-TARGETED SIRNA TO INHIBIT NONSENSE MEDIATED DECAY

(75) Inventors: Eli Gilboa, Coral Gables, FL (US); Fernando Pastor, Navarra (ES)

(73) Assignee: UNIVERSITY OF MIAMI, Miami, FL (US)

(21) Appl. No.: 13/380,464

(22) PCT Filed: Jun. 23, 2010
(86) PCT No.: PCT/US10/39626

§ 371 Date: Jun. 21, 2012

Related U.S. Application Data
(60) Provisional application No. 61/219,502, filed on Jun. 23, 2009.

(51) Int. Cl. C07H 21/02; A61K 39/00; A61P 37/04; C07K 9/00

(52) U.S. Cl. 424/184.1; 536/24.5; 530/322

(57) ABSTRACT

Compositions for inducing or enhancing antigenicity of a target cell by modulating the non-sense mediated decay pathway in the target cell. The compositions comprise one or more aplamers providing specificity and delivery of an oligonucleotide to the target, These compositions have broad applicability in the treatment of many diseases.

### CROSS-REFERENCE TO RELATED APPLICATIONS

The present application claims the priority of U.S. provisional patent application No. 61/219,502 filed Jun. 23, 2009, which is incorporated herein by reference in its entirety.

### FIELD OF THE INVENTION

Embodiments of the invention provide compositions and methods for highly selective targeting of heterologous nucleic acid sequences. The heterologous nucleic acid sequences comprise oligonucleotides, for example, short interfering RNA's (siRNA's) which are targeted to desired cells in vivo and which bind in a sequence dependent manner to their targets.

### BACKGROUND

Many therapeutic agents have been made available in the treatment of diseases such as cancer. There have been many challenges, however, in obtaining therapies which are effective. The important challenges facing many therapeutics in treatment of cancer include: (i) Metastatic disease, which is often undetectable and/or inaccessible, not the primary lesion, is the primary cause of death among cancer patients. The treatment, therefore, has to access disseminated disease, (ii) The drug has to be targeted to the tumor cells in the cancer patient. Expression of new products in nontransformed cells will expose normal tissue to the effects of the drug which can be toxic to the cells and the patient. (iii) The therapy should be clinically feasible, from the standpoint of cost, regulatory approval process, and complexity of treatment. For example, delivery and expression of “foreign” genes in tumor cells can be achieved using Adeno- or poxvirus-based vectors. However, poor tumor penetrance, their potential immunogenicity, and the challenges associated with using viral vectors in clinical settings, has precluded their use for this purpose. (iv) The therapy should be broadly useful for all types of cancer and cancer patients.

### SUMMARY

Embodiments of the invention comprise the generation of multi-domain molecules comprising a target specific domain and at least one domain, which modulates expression and function of molecules associated with nonsense mediated decay pathways.

In a preferred embodiment, the multi-domain molecules comprise aptamer-oligonucleotides for specifically targeting an oligonucleotide, e.g. interference RNA (RNAi) to a desired cell in vivo. The aptamers are generated against specific products expressed by a target cell and the oligonucleotides are specific for the nonsense mediated decay pathway and associated molecules. Inhibition of the nonsense mediated decay pathway allows for the up-regulation of existing antigens and/or the induction of new antigens not previously expressed on the target cells and/or novel antigens which results in the induction or enhancement of antigen city of a the target cell ultimately leading to its destruction by the immune system.

Methods of treating a patient comprise administration of a therapeutically effective amount of chimeric molecules, such as for example, aptamer-oligonucleotide molecules.

Other aspects are described infra.

### BRIEF DESCRIPTION OF THE DRAWINGS

FIG. 1 is a schematic representation showing the mechanism by which the NMD process prevents the accumulation of premature termination codon (PTC) containing mRNAs in eukaryotic cells. Removal of introns from the pre-mRNA leaves behind an exon-junction complex (EJC) demarcating the splice junctions (Panel A). An NMD complex consisting of several factors including Upf1, Upf2 and Up3 is then assembled on each EJC as shown in Panel B. SMG1, which phosphorylates Upf1, and Upf1 are the two key rate limiting factors in the formation of the complex. When the mRNA undergoes the first round of translation, called the “pioneer translation”, the EJC/NMD complex is removed, presumably as a result of the translational machinery moving thru the region, thereby rendering the mRNA stable and competent for additional rounds of translation. If a PTC is present in an exon (other than the last exon), for example as a result of an inframe nonsense mutation, the EJC/NMD complexes downstream to the FTC are not removed from the mRNA. The attached NMD complex then triggers the degradation of the mRNA.

FIG. 2 is a schematic representation showing the principle of targeted inhibition of NMD in tumor cells using aptamer targeted siRNAs. An aptamer which was selected to bind to a tumor-specific cell surface product, such as PSMA expressed on prostate cancer cells, is conjugated to an siRNA corresponding to an NMD factor such as SMG-1 or Upf2 (see below). The systemically administered aptamer-oligonucleotides homes to and delivers the siRNA to the tumor cells expressing the cognate receptor.

FIG. 3 shows that a PSMA aptamer—SMG-1 siRNA chimera downregulates SMG-1 RNA expression in PSMA-expressing tumor cells in a PSMA-dependent manner. Inhibition of SMG-1 RNA by PSMG-1 siRNA and PSMA apamer-SMG-1 siRNA chimera shows that conjugation of the siRNA to the aptamer did not compromise its functionality. Inhibition of SMG-1 RNA by the PSMA apamer-SMG-1 siRNA chimera in the absence of lipofectamine in PSMA-CT26 cells but not CT26 cells shows that the SMG-1 siRNA was targeted to CT26 cells in a PSMA-dependent manner.

FIGS. 4A-4C show the expression of Upf2 or Smg1 shRNA in CT26 tumor cells leads to immune-mediated inhibition of tumor growth. FIG. 4A: Intratumoral accumulation of OVA-specific OT-I T cells in response to NMD inhibition. B16/F10 tumour cells transduced with shRNA-encoding lentiviral vectors (described in FIG. 8A) were stably transfected with an NMD reporter plasmid (described in FIG. 8B) containing the class I-restricted epitope of chicken ovalbumin (OVA). Mice were implanted subcutaneously with parental tumor cells (wild-type (WT) B16) or with the lentivirus-transduced tumor cells, arnd either received or did not receive doxycycline in their drinking water. When tumors became palpable, mice were injected with either OT-I or Pmel-1 transgenic CD8⁺ T cells (three mice per group). Six days later, tumors were excised and analyzed for OT-1 and Pmel-1 T-cell content by flow cytometry. Ctrl, control, n=2. FIG. 4B: Balb/c mice were implanted subcutaneously with CT26 tumor cells stably transduced with the shRNA inducible lentiviral vector encoding Smg1, Upf2 and control shRNA (ten mice per group). Each group was divided into two subgroups receiving (filled circles) or not receiving (open circles) doxycycline in the drinking water, n=2. FIG. 4C: Same as FIG. 4B except that tumor cells were injected into immune-deficient nude mice. n=1.

FIGS. 5A-5C are graphs showing the inhibition of tumor growth in mice treated with PSMA aptamer targeted Upf2 and Smg1 siRNAs. FIG. 5A: Balb/c mice were implanted subcutaneously with PSMA-CT26 tumor cells and 3 days later injected via the tail vein with PBS (filled circles) or with PSMA aptamer-siRNA conjugates (open circles, control siRNA; open squares, Upf2 siRNA; filled squares, Smg1 siRNA) (5 mice per group), n=2. FIG. 5B: C57BL/6 mice were implanted with PSMA-B16/F10 tumor cells by tail vein injection, and 5 days later were injected with PSMA aptamer-siRNA conjugates (ten mice per group). Metastatic load was determined by measuring lung weight at the time of euthanization, n=2. FIG. 5C: Combination immunotherapy using NMD inhibition and 4-1BB co-stimulation. PSMA-CT26 tumor-bearing mice (five mice per group) were treated with various combinations of PSMA aptamer conjugated to Smg1 or control siRNA and an agonistic or costimulation-deficient 4-1BB aptamer dimer (mut4-1BB) and monitored for tumor growth. n=1.

FIGS. 6A-6C: PSMA aptamer-Smg1 siRNA rejection of PSMA-expressing, but not parental, CT26 tumor cells. FIG. 6A: Mice were co-implanted subcutaneously with PSMA-expressing (left flank) and parental (right flank) CT26 tumor cells and injected with PSMA aptamer-Smg1 siRNA via the tail vein. FIG. 6B: Fifteen days after tumor inoculation, ³²P-labeled aptamer-siRNA was injected, and 3 or 24 h later tumors were excised and the ³²P content determined. n=3. FIG. 6C: Three days after tumor inoculation, mice were injected with aptamer-siRNA conjugate (eight mice per group) as described in FIG. 5A and tumor growth was monitored. Open circles, parental CT26; filled circles, PSMA-CT26. n=2.

FIG. 7: Comparison of PSMA aptamer-Smg1 siRNA treatment to vaccination with GM-CSF expressing irradiated tumor cells. C57BL/6 mice were injected intravenously with B16/F10 tumor cells and treated with PSMA aptamer-siRNA conjugates starting at day 5 as described in FIG. 5C, or vaccinated with GM-CSF expressing irradiated B16/F10 tumor cells (GVAX) starting at days (D) 1 or 5. n=1.

FIGS. 8A-8B: RNA downregulation and NMD inhibition in CT26 tumor cell stably expressing Upf-2 or SMG-1 shRNA. Colon carcinoma-derived CT26 tumor cells were stably transduced with the PTIG-U6tetO lentiviral vector encoding Upf-2 and SMG-1 shRNA. PTIG-U6tetOshRNA contains a U6-promoter driven shRNA cassette which is under tet regulation (Diagram, FIG. 8A), as well as a bicistronic CMV promoter-driven cassette encoding the tet repressor and EGFP) (not shown in the diagram). Stably transduced cells were isolated by sorting for EGFP⁺ cells and grown in the presence or absence of doxycycline. FIG. 8A: Expression of Upf-2 or SMG-1 RNA is reduced in CT26 tumor cells expressing the corresponding shRNAs (culturing tumor cells in the presence of doxycycline), but not parental CT26 tumor cells. The relative amounts of actin, Up12 and SMG-1 RNA were determined by semi-quantitative RT-PCR using limited cycles of amplification. FIG. 8B: shRNA-mediated Upf-2 or SMG-1 downregulation leads to NMD inhibition. NMD activity in the shRNA-expressing CT26 cells was determined by transiently transfecting the Upf-2 and SMG-1 shRNA expressing CT26 cells with an NMD reporter plasmid encoding a β-globin gene which contains a premature termination codon (PTC) in its second exon (see diagram) or with a similar plasmid encoding the wild type (wt) β-globin gene. Cells were grown in the presence or absence of doxycycline and the relative amounts of β-globin transcripts were determined by semi-quantitative RT-PCR.

Cells transfected with the PTC-containing, but not wild type, β-globin gene accumulated reduced levels of globin transcripts due to NMD. However, when Upf-2 or SMG-1 shRNA expression was induced by growing cells in the presence of doxycycline leading to downregulation of its RNA as shown in the upper panel, expression of the PTC-containing globin transcripts is upregulated, consistent with inhibition of NMD.

FIG. 9: Delaying SMG-1 shRNA expression in tumor bearing mice diminishes tumor inhibition. Balb/c mice were implanted subcutaneously with the shRNA inducible lentiviral vector encoding SMG-1 shRNA as described in FIG. 4B. As indicated, doxycycline was provided in the drinking water at the day of tumor implantation (day 0) or delayed for 2, 4 or 6 days (5 mice per group).

FIGS. 10A-10C: Induction of T cell responses against NMD-controlled products. Mice were immunized with CT26 tumor cells transduced with the doxycycline-inducible SMG-1 (SMG-1) or control (Control) shRNA (FIG. 8A) in the presence doxycycline in the drinking water, and 5 or 30 days later splenocytes were isolated and enriched for either total CD3⁺ T cells, CD8⁺ cells, or CD4⁺ T cells. FIG. 10A: Induction of immune responses against tumor cells in which NMD was inhibited. Tumor antigens in the form of mRNA were isolated from SMG-1 (SMG-1) or control (Control) shRNA transduced CT26 tumor cells cultured in the presence of doxycycline, and transfected into syngeneic dendritic cells (DC). T cells isolated 5 days after tumor implantation (responders) were incubated with the mRNA transfected DC (stimulators), and proliferation was determined 4 days later by measuring ³H-thymidine incorporation. Only T cells from mice immunized with NMD-inhibited tumor cells (doxycycline in the drinking water) stimulated with DC transfected with mRNA derived from tumor cells in which NMD was inhibited (cultured in the presence of doxycycline) exhibited statistically significant enhanced proliferation, FIG. 10B. No induction of immune responses against normal tissues. Total CD3⁺ T cells from mice immunized with CT26 tumor cells stably expressing SMG-1 shRNA (doxycycline in the drinking water) were incubated with DC transfected with mRNA isolated from liver, colon and prostate (Control—no mRNA) and proliferation was measured. (Note, the use of mRNA transfected DC as stimulators provides a useful way to compare the antigenicity of diverse cell populations and tissues that often exhibit significant variations in their background immunogenicity, e.g., stimulation of T cell proliferation measured by ³Hthymidine incorporation.) FIG. 10C, Epitope spread—induction of immune responses against the parental tumor. Experimental conditions as described in FIG. 10A using total CD3⁺ T cells as responders, except that T cells were isolated 5 day as well as 30 days post tumor implantation.

FIG. 11 is a schematic representation showing the primary sequence and computer generated secondary structure of an embodiment of a PSMA aptamer-siRNA conjugates. PSMA aptamer-siRNA fusions were generated corresponding to the SWAP configuration except that two thymidine nucleotides were added to the 3′ of the passenger strand to prevent dicer binding. RNAstructre 4.1 program was used for secondary structure analysis, PSMA aptamer—black; siRNA guide strand—blue; siRNA passenger strand—red.

FIG. 12: Binding and uptake of PSMA aptamer-SMG-1 siNA by PSMA-expressing CT26 tumor cells. Parental and PSMA-expressing CT26 tumor cells were incubated with anti-PSMA antibody (green) or Cy3-conjugated PSMA aptamer-SMG-1 siRNA (pink) and analyzed by confocal microscopy (60× magnification). Nuclei were stained with DAPI (blue).

FIG. 13. PSMA-dependent inhibition of SMG-1 or Upf-2 RNA in PSMA-CT26 tumor cells incubated with PSMA aptamer-siRNA conjugates. PSMA-C-T26 or parental CT26 cells were incubated with SMG-1 siRNA, control siRNA, PSMA aptamer-SMG-1 siRNA or PSMA aptamer-control siRNA (left panel), or with PSMA aptamer-Upf-2 siRNA and PSMA-control siRNA (right panel), in the presence or absence of lipofectamine, as shown. SMG-1 and Upf-2 RNA content was determined 48 hours later using semiquantitative RT-PCR. The SMG-siRNA retains its function when conjugated to the PSMA aptamer since free and conjugated siRNA transfected in the presence of lipofectamine were comparably effective in down-regulating SMG-1 RNA (left panel), Inhibition by the aptamer conjugated siRNAs is PSMA dependent since in the absence of transfection agent, incubation of PSMA-expressing, but not parental, CT26 tumor cells with either PSMA aptamer-SMG-1 or Upf-2 siRNA conjugates led to downregulation of its target RNA.

FIGS. 14A-14B: Inhibition of tumor growth and survival of CT26 tumor bearing mice injected with PSMA-SMG-1 siRNA conjugates. Balb/c mice were implanted subcutaneously with PSMA-CT26 tumor cells and 3 days later injected via the tail vein with PBS, PSMA aptamer-control siRNA conjugate (Con), or with PSMA aptamer-SMG-1 siRNA conjugate. PSMA aptamer-SMG-1 siRNA conjugate was administered at a dose of 400 pmoles per injection on days 3, 5, 7, 9, 11, and 13, (SMG-1 (1×), or at a dose of 800 pmoles per injection administered at days 3, 5, 7, 9, 11, 13, 15 and 17, (SMG-1 (2×). FIG. 14A: Survival. The long term surviving mice (>40 days) had no evidence of tumor. Statistical analysis: SMG-1 (1×) versus PBS, p=0.0002; SMG-2 (2×) versus SMG-1, p=0.0327. FIG. 14B. Tumor size.

FIG. 15: PSMA aptamer-SMG-1 siRNA rejection of PSMA-expressing, but not parental, CT26 tumor cells—Tumor size at day of sacrifice. Mice were sacrificed at day 19 when tumors in the PBS group reached maximum allowable size (12 mm diameter). Only the PSMA-CT26 tumors in mice treated with PSMA-SMG-1 siRNA have shown consistently diminished growth compared to contralaterally implanted parental CT26 tumors; in this experiment in 7 out of 8 mice the PSMA-CT26 tumors were either smaller than the parental CT26 tumors or completely regressed.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention, Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” arnd “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochenm. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

As used herein, the term “multi-domain molecules” refers to the different variations of the therapeutic molecules that comprise a domain which specifically targets or delivers the molecule to a desired cell or in vivo locale and a second domain which modulates expression or function of the nonsense mediated decay pathway or molecules associated with these pathways. For example, the multi-domain molecule can comprise at least one aptamer conjugated, linked, fused, etc., to an oligonucleotide such as a siRNA, which modulates the function of the NMD pathway and/or the expression and function of a molecule associated with the NMD pathway.

As used herein, the term “aptamer-oligonucleotide” refers to the compositions described herein wherein at least one aptamer is linked or conjugated to at least one antisense oligonucleotide. Combinations of more than one aptamer and oligonucleotides, with more than one specificity, are included.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the term “oligonucleotide,” is meant to encompass all forms or desired RNA, RNA/DNA molecules which modulate gene expression and/or function, and includes without limitation: “siRNA,” “shRNA” “antisense oligonucleotide” etc. The term also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoögsteen types of base pairing, or the like.

The aptamer-oligonucleotide may be “chimeric,” that is, composed of different regions. In the context of this invention “chimeric” compounds are aptamer-oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphonates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynycytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0]bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of for example, a certain population with a propensity for a disease state, that is susceptibility versus resistance.

As used herein, the term “mRNA” means the presently known mRNA transcript(s) of a targeted gene, and any further transcripts which may be elucidated.

By “desired RNA” molecule is meant any foreign RNA molecule which is useful from a therapeutic, diagnostic, or other viewpoint. Such molecules include antisense RNA molecules, decoy RNA molecules, enzymatic RNA, therapeutic editing RNA and agonist and antagonist RNA.

By “antisense RNA” is meant a non-enzymatic RNA molecule that binds to another RNA (target RNA) by means of RNA-RNA interactions and alters the activity of the target RNA (Eguchi et al., 1991 Annu. Rev. Biochem. 60, 631-652).

RNA interference “RNAi” is mediated by double stranded RNA (dsRNA) molecules that have sequence-specific homology to their “target” nucleic acid sequences (Caplen, N. J., et al., Proc. Natl Acad. Sci. USA 98:9742-9747 (2001)). In certain embodiments of the present invention, the mediators of RNA-dependent gene silencing are 21-25 nucleotide “small interfering” RNA duplexes (siRNAs). The siRNAs are derived from the processing of dsRNA by an RNase enzyme known as Dicer (Bernstein, E., et al., Nature 409:363-366 (2001)). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al., Nature 409:363-366 (2001); Boutla, A., et al., Curr. Biol. 11:1776-1780 (2001)). Small interfering RNAs that can be used in accordance with the present invention can be synthesized and used according to procedures that are well known in the art and that will be familiar to the ordinarily skilled artisan, Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

By “enzymatic RNA” is meant an RNA molecule with enzymatic activity (Cech, 1988 J. American. Med. Assoc. 260, 3030-3035). Enzymatic nucleic acids (ribozymes) act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through base-pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is meant to be a specific example. Those in the art will recognize that this is but one example, and other embodiments can be readily generated using techniques generally known in the art.

The term, “complementary” means that two sequences are complementary when the sequence of one can bind to the sequence of the other in an anti-parallel sense wherein the 3′-end of each sequence binds to the 5′-end of the other sequence and each A, T(U), (G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. As the target sequences are generally present in excess, at Tm, 50% of the oligonucleotides are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

The term “stringent conditions” refers to conditions under which an oligonucleotide will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 1-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3^{rd} ed. Cold Spring Harbor Laboratory Press: Cold Spring Harbor, N.Y.; Sambrook et al., (1989) Molecular Cloning: A Laboratory Manual. 2^{nd} ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

### Compositions

Disseminated metastatic disease is the primary cause of death among cancer patients. Cancer vaccination stimulates a systemic immune response against judiciously chosen tumor antigens expressed in the tumor cells that seeks out and destroys the disseminated tumor lesions. The development of effective cancer vaccines will require the identification of potent and broadly expressed tumor rejection antigens (TRAs) (Gilboa, E. Immunity 11, 263-270 (1999); Novellino, L., et al. Cancer Immunol Innunother. 54, 187-207 (2005); Schietinger, A., et al. Semin. immunol. 20, 276-285 (2008)) as well as effective adjuvants to stimulate a robust and durable immune response (Gilboa, E. Nature Rev. Cancer 4, 401-411 (2004); Melief, C. J. Immunity 29, 372-383 (2008); Pardoll, D. M. Nature Rev. Immunol. 2, 227-238 (2002)).

Alternative novel approaches to vaccination are provided herein. Embodiments of the invention comprise expressing new, and hence potent, antigens in tumor cells in situ. How to express new antigens in the disseminated tumor lesions, but not in normal tissue, have heretofore precluded the development of such strategies.

Nonsense mediated decay (NMD) prevents the expression of aberrant products in the cell. In preferred embodiments, inhibition of NMD in tumor cells leads to the expression of novel antigens and enhancement of the immunogenicity of tumor cells, leading to tumor rejection by the immune response. Delivery of siRNA targeted to NMD pathways in vivo can be used to inhibit NMD. However, non-targeted delivery of siRNA in vivo is not clinically practical because of cost consideration and anticipated toxicity. Targeting siRNA to the appropriate cells, tumor cells in this instance, would solve the problem. Currently antibodies are the obvious choice for targeting ligands. However, since antibodies are cell based products, and hence pose significant cost, manufacturing, and regulatory challenges.

Developing a clinically feasible and generally applicable method to enhance the antigenicity of tumors in situ will have major impact on controlling cancer. The targeting strategy, described in embodiments herein—use of oligonucleotide-based aptamers—is a novel platform technology that can be used to develop improved methods of delivering therapeutic cargo such as small MW drugs, toxins, siRNAs, as well as therapeutic aptamers, to cells in vivo.

In addition, the compositions described herein, provide novel methods of in vivo drug targeting that offers potential advantages over the use of antibodies and protein-based therapeutic agents as targeting ligands.

In general, the invention provides compositions and methods for inhibiting nonsense-mediated mRNA decay (NMD) and/or a component of the NMD pathway in a cell. Embodiments of the invention are directed to a clinically useful approach to express new antigens in the disseminated tumor lesions of cancer patients by targeted inhibition of nonsense mediated decay (NMD) in the tumor cells. NMD is an evolutionary conserved mRNA surveillance pathway in eukaryotic cells that detects and eliminates mRNAs harboring premature termination codons (PTCs). Without wishing to be bound by theory, the central hypothesis is that upregulation of gene expression when NMD is inhibited in tumor cells will translate into therapeutically useful enhancement of tumor antigenicity, namely that the new products will function as effective tumor antigens, capable of eliciting an immune response which will contribute to tumor rejection. Inhibition will be accomplished using siRNAs against NMD factors which will be targeted to tumor cells or any other abnormal cell by conjugation to oligonucleotide (ODN)-based aptamer ligands. Thus, the targeted NMD inhibitory agent comprises at least a single chemically synthesized oligonucleotide molecule.

In a preferred embodiment, a composition for inducing novel antigens in abnormal cells, comprises a multi-domain molecule having at least one target specific domain and at least one domain, which modulates expression and function of molecules associated with nonsense mediated decay pathways.

In another preferred embodiment, the multi-domain molecule comprises at least one target specific domain and at least two domains which modulate expression and function of one or more molecules associated with nonsense mediated decay pathways. Examples of such molecules comprise: RENT1, RENT2, eIF4A, UPF1, UPF2, UPF3BI, RNPS1, Y14, MAGOH, NMD1, SMG, or combinations thereof.

In another preferred embodiment, the multi-domain molecule comprises at least two target specific domains and at least one domain which modulates expression and function of one or more molecules associated with nonsense mediated decay pathways.

In another preferred embodiment, the target specific domains comprise specificities for similar target molecules, different target molecules, or combinations thereof.

In another preferred embodiment, the domains which modulate expression and function of one or more molecules associated with nonsense mediated decay pathways modulate the expression and function of similar targeted molecules, different targeted molecules or combinations thereof.

In another preferred embodiment, the target specific domains are specific for target cell molecules, the target cell comprising: a tumor cell, an infected cell, a tissue specific cell, an adipocyte, a stem cell, an immune cell, an organ specific cell or a transformed cell.

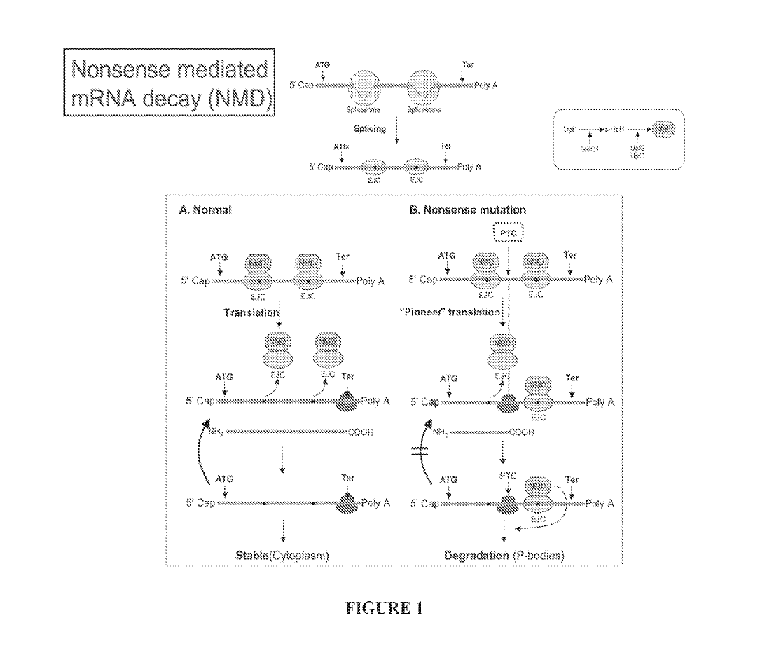

Nonsense mediated (mRNA) decay (NMD): FIG. 1 is a schematic representation showing the mechanism by which the NMD process prevents the accumulation of PTC containing mRNAs in eukaryotic cells. In brief, removal of introns from the pre-mRNA leaves behind an exon-junction complex (EJC) demarcating the splice junctions (Panel A). An NMD complex consisting of several factors including Upf1, Upf2 and Up3 is then assembled on each EJC as shown in Panel B. SMG1, which phosphorylates Upf1, and Upf1 are the two key rate limiting factors in the formation of the complex. When the mRNA undergoes the first round of translation, called the “pioneer translation”, the EJC/NMD complex is removed, presumably as a result of the translational machinery moving through the region, thereby rendering the mRNA stable and competent for additional rounds of translation. If a PTC is present in an exon (other than the last exon), for example as a result of an inframe nonsense mutation, the EJC/NMD complexes downstream to the PTC are not removed from the mRNA. The attached NMD complex then triggers the degradation of the mRNA.

In preferred embodiments, a composition comprising oligonucleotides directed against NMD-specific factors inhibit NMD in tumor cells (see, for example, FIG. 1). The siRNAs targeted to the tumor by conjugation to aptamer-based ligands. Targeting the siRNAs to tumor cells is a preferred method for the therapeutic use of this approach since upregulation of new products in nontransformed cells could expose normal tissue to immune destruction creating an “autoimmune inferno”.

NMD-mediated degradation of mRNA is not limited to instances of mutations or recombinations generating PTCs. Error-free mRNAs containing short 5′ open reading frames (ORFs). mRNAs which are regulated by stop codon read-through, leaky scanning for translation initiation, or regulated frameshifting or mRNA can be also recognized by NMD.

Physiological roles of NMD: It was initially thought that the main role of NMD was to maintain the proteome integrity of the cell by eliminating transcripts with nonsense mutations generating PTCs yielding truncated products. Indeed, over 30% of genetic disorders are caused by PTC. In several instances the severity of the disease, e.g. β-thalassemia, correlates with the NMD-controlled degradation of the mutant mRNA. Yet, nonsense mutations generating PTCs are rare events and it is unlikely that the NMD system has evolved to counter their potential deleterious effects. There is in fact accumulating evidence that the main and physiological role of the NMD is to regulate normal gene expression.

An important role of NMD is to maintain splicing integrity. The efficiency and accuracy of splicing is notoriously imperfect. Such transcripts will often contain PTCs and hence become targets for NMD elimination. NMD is also responsible for the elimination of transcripts encoding nonproductively rearranged T cell receptors and immunoglobulin chains. A significant proportion of gene products (>15%) that are upregulated when NMD is inhibited, such as by targeting Upf1 with siRNA are involved in amino acid biosynthesis and transcription factors which coordinate cellular responses to starvation. Since starvation also down-regulates translation thru phosphorylation and inhibition of eIF2a, which in turn inhibits NMD efficiency, it appears that the response to starvation is in part under NMD control. NMD is also implicated in several instances of products autoregulating alternative splicing (e.g., serine-arginine (SR)-rich proteins and hnRNP splicing factors such as SC35, calpain, CDC-like kinases, biosynthesis of selenoproteins, and telomere synthesis.

Other functions of Upf1 and SMG1. In preferred embodiments, a method of inducing or enhancing tumor antigenicity comprises a composition having an aptamer specific for a target cell conjugated to an agent which inhibits NMD in tumor cells, for example, siRNAs specific for key NMD factors. Upf1 also promotes the replication-dependent decay of histone mRNA which is required for cell cycle progression. SMG1 is a kinase which also phosphorylates and inactivates p53; siRNA inhibition of SMG-1 in U2OS cells results in the accumulation of dsDNA break and activation of ATM- or ATR-mediated checkpoint responses. Both Upf1 and SMG1 were implicated in telomere maintenance by facilitating the binding of telomere repeat-containing RNA (TERRA) to telomeres.

Cancer cells accumulate elevated level of PTC containing NMD mRNA substrates. About 15% of cancers exhibit defects in DNA mismatch repair (MMR) often manifested as microsatellite instability (MSI). Such defects affecting many products, including products associated with tumor progression such as TGFβRII, APAF-1, IGFIIR, BAX, PTEN, RHAMM, give rise to frameshift mutation ending in PTCs. Such PTC-containing transcripts are under NMD control whereby Upf1 siRNA mediated inhibition of NMD in a human colorectal cancer cell line exhibiting an MSI phenotype stabilized the frameshifted mutant transcripts. Such products could provide a source of tumor-specific antigenic determinants downstream the recombination site. Thus, increased immune infiltrate are seen in tumors with MIS phenotype would correlate with the levels of Upf1 in the tumors. Inhibiting NMD further augments the production of such tumor-specific antigens.

Aptamer mediated targeting of siRNA: Targeting the siRNAs to tumor cells is a preferred embodiment. The methods utilize the following approach: (i). Upregulation of new products in nontransformed cells would expose normal tissue to immune destruction creating an “autoimmune inferno.” (ii). NMD is a physiologically important process regulating various house-keeping functions of somatic cells and the key factors of the NMD process, SMG1 and Upf1, play also important roles in maintaining genome stability and cells survival. Inhibiting NMD in somatic cells would be, deleterious. (iii). Targeting the oligonucleotide-based aptamer-oligonucleotides agent to tumor cells will reduce the cost of treatment and the risk of adverse effects associated with the non-specific stimulatory properties of nucleic acids.

Monoclonal antibodies have been used as ligands with engineered specificity to target drugs, toxins as well as siRNAs to cells. A major limitation of using antibodies in therapeutic settings is limited, and at best uncertain, access to this class of biologicals. The reason is that antibodies are cell-based products posing significant cost, manufacturing and regulatory challenges. Hence, clinical-grade reagents are almost exclusively developed and provided by companies on a selective basis and under strict contractual agreement.

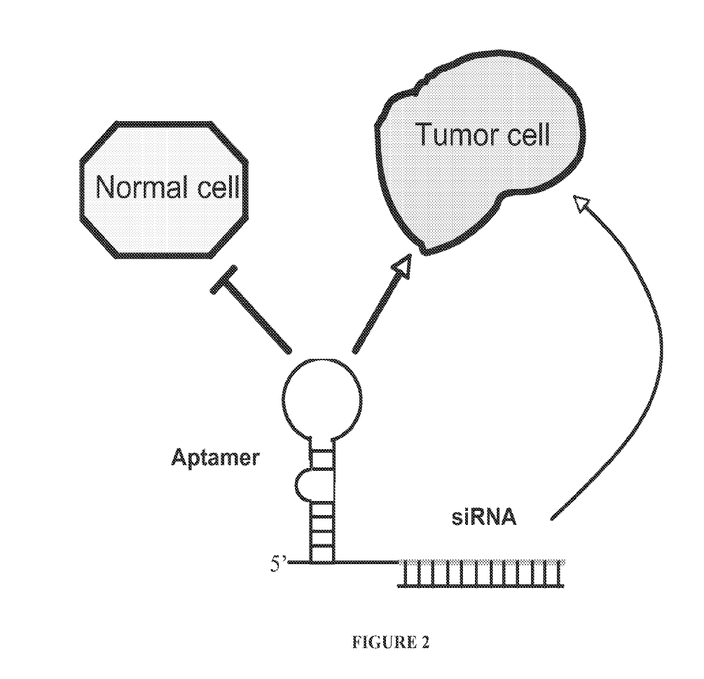

In contrast, aptamers are high affinity single stranded nucleic acid ligands which can be isolated trough a combinatorial chemistry process known as SELEX. Aptamers with nuclease-resistant backbone can be generated against most targets, proteins as well as small molecules, and exhibit remarkable affinity and specificity to their targets comparable to and often exceeding that of antibodies. Importantly, and what is a key advantage of aptamers, the 25-40 nt long aptamers can be synthesized chemically. Consequently, manufacture of clinical grade aptamers, including aptamer-oligonucleotides fusion ODNs, is relatively cost effective, and the regulatory approval process significantly simpler. FIG. 2 shows how aptamers can be used to target siRNAs to tumor cells.

As used herein, “an aptamer” is inclusive of one or more aptamers that may have the same specificity for a target molecule, or the aptamers are specific for different targets. Thus, when using the term “aptamer” the term applies to one or a plurality of aptamers linked or conjugated together and can each be specific for different target antigens.

In a preferred embodiment, the gene silencing agent (the RNAi) is targeted to the appropriate cells in vivo using nuclease-resistant oligonucleotide-based aptamers. Targeting of polynucleotides, without limitation, any one or more components of a nonsense mediated decay pathway. The display of new antigens or an increase in antigens that can be recognized by the immune system as abnormal or foreign results in the destruction of that cell.

In another preferred embodiment, a composition comprising a targeting agent and a gene silencing agent down-regulate or abrogate nonsense mediated decay pathways. In a preferred embodiment, the gene silencing agent is an RNAi (siRNA/shRNA).

As an illustrative example, siRNAs to SMG-1, Upf2 and Upf3 were characterized and stably transduced CT26 tumor cells with a lentivirus-based vector (LV) expressing the siRNAs from a tet-inducible U6 promoter. Having confirmed that doxycycline treatment induced siRNA expression and NMD inhibition in the culture cells, measured as downregulation of the corresponding mRNA by semiquantitative RT-PCR and stabilization of a PTC-containing mRNA expressed from an NMD reporter plasmid, the tumor cells were implanted in mice and tumor growth was monitored in the presence or absence of doxycycline, siRNA inhibition of SMG-1 or Upf2 led to an almost complete inhibition of tumor growth. Inhibition of Upf3 was significant but less pronounced while expression of a control siRNA had no effect. No evidence direct of toxicity, viability or proliferative capacity, was seen in the siRNA-expressing tumor cells cultured for two weeks in the presence of doxycycline.

In a preferred embodiment, the siRNA, antisense oligonucleotides are directed to factors associated with the NMD pathway comprising at least one of: RENT1, RENT2, eIF4A, UPF1, UPF2, UPF3B, RNPS1, Y14, MAGCOH, NMD1 or SMG.

In another preferred embodiment, the aptamer-oligonucleotide molecule can be administered in conjunction with one or more agents which inhibit NMD. For example, use of pharmacological agents that inhibit protein translation. Examples of such drugs are described in Noensie and Dietz ((2001) Nature Biotech 19: 434-439), the contents of which are incorporated herein by reference. This approach is based upon the finding that NMD is generally inhibited by agent that block or inhibit protein translation. Examples of such agents include emetine, anisomycin, cycloheximide, pactamycin, puromycin, gentamicin, neomycin, and paromomycin. Other protein translational inhibitors are known in the art and may be utilized in the method of the invention (see e.g. Leviton (1999) Cancer Invest 17: 87-92 (inhibitors of protein synthesis); and Bertram (2001) Microbiology 147: 255-69 (detailed description of the molecular biology of protein translation)).

Other Aptamer-Composition Permutations: In a preferred embodiment of the invention, a nucleic acid is associated with the aptamers. The nucleic acid can be selected from a variety of DNA and RNA based nucleic acids, including fragments and analogues of these. A variety of genes for treatment of various conditions have been described, and coding sequences for specific genes of interest can be retrieved from DNA sequence databanks, such as GenBank or EMBL. For example, polynucleotides for treatment of viral, malignant and inflammatory diseases and conditions, such as, cystic fibrosis, adenosine deaminase deficiency and AIDS, have been described. Treatment of cancers by administration of tumor suppressor genes, such as APC, DPC4, NF-1, NF-2, MTS1, RB, p53, WT1, BRCA1, BRCA2 and VHL, are contemplated.

Examples of specific nucleic acids for treatment of an indicated conditions include: HLA-B7, tumors, colorectal carcinoma, melanoma; IL-2, cancers, especially breast cancer, lung cancer, and tumors; IL-4, cancer; TNF, cancer; IGF-1 antisense, brain tumors; IFN, neuroblastoma; CM-CSF, renal cell carcinoma; MDR-1, cancer, especially advanced cancer, breast and ovarian cancers; and HSV thymidine kinase, brain tumors, head and neck tumors, mesothelioma, ovarian cancer.

The polynucleotide can be an antisense DNA oligonucleotide composed of sequences complementary to its target, usually a messenger RNA (mRNA) or an mRNA precursor. The mRNA contains genetic information in the functional, or sense, orientation and binding of the antisense oligonucleotide inactivates the intended mRNA and prevents its translation into protein. Such antisense molecules are determined based on biochemical experiments showing that proteins are translated from specific RNAs and once the sequence of the RNA is known, an antisense molecule that will bind to it through complementary Watson-Crick base pairs can be designed. Such antisense molecules typically contain between 10-30 base pairs, more preferably between 10-25, and most preferably between 15-20.

The antisense oligonucleotide can be modified for improved resistance to nuclease hydrolysis, and such analogues include phosphorothioate, methylphosphonate, phosphodiester and p-ethoxy oligonucleotides (WO 97/07784).

The aptamer can be specific for any type of products, for example, tumor antigens, cell adhesion molecules, such as for example, integrins, glycosylated proteins, etc. For example, cell adhesion molecules, such as integrins, play a vital role in angiogenesis, a key pathway for tumor growth, invasion and metastasis. RGD is an alternative to aptamer—it targets “cargo” to inflammed endothelial cells.

Other examples comprise: stromal derived factor 1 (SDF-1), MCP-1, MIP-1α, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, MAdCAM-1, α4 integrins, α5 integrins, beta defensins 3 and 4.

The target molecule can be one that binds to, for example, an extracellular domain of a growth factor receptor. Exemplary receptors include the c-erbB-2 protein product of the HER2/neu oncogene, epidermal growth factor (EGF) receptor, basic fibroblast growth receptor (basic FGF) receptor and vascular endothelial growth factor receptor, E-, L- and P-selectin receptors, folate receptor, CD4 receptor, CD19 receptor, α, β-integrin receptors and chemokine receptors.

In other preferred embodiments, the aptamers may also be conjugated to transporter proteins to increase the transportation of the oligonucleotides specific for NMD factors across membranes e.g. blood brain barrier, intestines, etc.

A “chemotherapeutic agent” which can also be the cargo moiety for treatment of a tumor is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedepa, and uredopa; ethylenimines and methylamelamines including altretamine, triethylenemelamine, trietylenephospsphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The cargo moiety delivered in association with the aptamer included in an inventive system may be any of various therapeutic and diagnostic agents which are desired to be delivered to a target. Therapeutic agents which can be included as cargo moieties in the delivery system of the present invention illustratively include but are not limited to therapeutic compounds such as an analgesic, an anesthetic, an antibiotic, an anticonvulsant, an antidepressant, an antimicrobial, an anti-inflammatory, anti-migraine, an antineoplastic, an antiparasitic, an antitumor agent, an antiviral, an anxiolytic, a cytostatic, cytokine, a hypnotic, a metastasis inhibitor, a sedative and a tranquilizer.

In another preferred embodiment, the aptamers are labeled with a detectable agent, which are administered to a patient for the in vivo imaging of a tumor. The specific delivery of the detectable agent provides a vastly superior means of specific detection of a tumor or desired target cell and decreases any background noise, allowing for the early detection and diagnosis of, for example, a tumor.

Diagnostic agents that may be included in the delivery system of the present invention as cargo moieties illustratively include but are not limited to a contrast agent, a labeled imaging agent such as a radiolabeled imaging agent, and an antitumoral agent. Combinations of therapeutic compounds may be included, combinations of diagnostic agents may be included, and combinations of both therapeutic and diagnostic agents may be included. Further suitable therapeutic and diagnostic compounds that may be delivered by a system according to the invention may be found in standard pharmaceutical references such as A, R. Gennaro, Remington: The Science and Practice of Pharmacy, Lippincott Williams & Wilkins, 20th ed. (2003); L. V. Allen, Jr. et al., Ansel's Pharmaceutical Dosage Forms and Drug Delivery Systems, 8th Ed. (Philadelphia, Pa.: Lippincott, Williams & Wilkins, 2004); J. 0. Hardman et al., Goodman & Gilmans The Pharmacological Basis of Therapeutics, McGraw-Hill Professional, 10th ed. (2001).

In another preferred embodiment, the detectable agent which is associated with the aptamer can also be a therapeutic agent. For example, a radioactive material which can be utilized as an imaging agent, and at the same time is a therapeutic agent when it is delivered locally to the tumor by the aptamer specific for that cell.

In another preferred embodiment, the aptamer is conjugated to a diagnostic agent and a therapeutic moiety.

Feasibility, generality, and potential of using aptamer targeted siRNA/gene silencing to modulate antitumor immunity by inducing or enhancing antigenicity of target cell: The use of aptamer-oligonucleotides to manipulate tumor immunity is directed to increase the expression of existing antigens or induce expression of novel antigens which would be recognized by the immune system as foreign. Use of aptamers to target gene silencing to the appropriate cells in vivo provides a drug/reagent that can be chemically synthesized in cell-free systems which significantly enhances the clinical applicability of this targeting approach (compared to antibody-based targeting), drastically reducing the amount of siRNA reagent needed for treatment and consequently the cost-effectiveness and toxicity of the treatment. Furthermore, a key advantage of immune modulating drugs, whether targeted or not, is that only a fraction of the target cells need to be accessed in vivo for the approach to be successful.

### Generation of Oligonucleotides:

Detailed methods of producing the RNAi's are described in the examples section which follows. The RNAi's of the invention can also be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, the RNAi's of the invention are chemically synthesized using appropriately protected ribonucleotide phosphoramidites and a conventional DNA/RNA synthesizer. The RNAi can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, RNAi can also be expressed from recombinant circular or linear DNA plasmids using any suitable promoter. Suitable promoters for expressing RNAi of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the RNAi in a particular tissue or in a particular intracellular environment. RNAi's of the invention can be expressed from a recombinant plasmid either as two separate, complementary RNA molecules, or as a single RNA molecule with two complementary regions.

Selection of plasmids suitable for expressing RNAi of the invention, methods for inserting nucleic acid sequences for expressing the RNAi into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16:948-958; Lee N S et al, (2002), Nat. Biotechnol. 20: 500-505; and Paul C P et al. (2002), Nat. Biotechnol. 20: 505-508, the entire disclosures of which are herein incorporated by reference.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

Any viral vector capable of accepting the coding sequences for the siRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the RNAi into the vector, and methods of delivering the viral vector to the cells of interest are within the skill in the art. See, for example, Dornburg R (1995), Gene Therap. 2: 301-310; Eglitis M A (1998), Biotechniques 6: 608-614; Miller A D (1990), Hum Gene Therap. 1: 5-14; and Anderson W F (1998), Nature 392: 25-30, the entire disclosures of which are herein incorporated by reference.

A suitable AV vector for expressing the RNAi's of the invention, a method for constructing the recombinant AV vector, and a method for delivering the vector into target cells, are described in Xia H et al. (2002), Nat. Biotech. 20: 1006-1010. Suitable AAV vectors for expressing the RNAi's of the invention, methods for constructing the recombinant AAV vector, and methods for delivering the vectors into target cells are described in Samulski R et al. (1987), J. Virol. 61: 3096-3101; Fisher K J et al. (1996), J. Viol., 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosure of which are herein incorporated by reference.

The ability of an RNAi containing a given target sequence to cause RNAi-mediated degradation of the target mRNA can be evaluated using standard techniques for measuring the levels of RNA or protein in cells. For example, RNA of the invention can be delivered to cultured cells, and the levels of target mRNA can be measured by Northern blot or dot blotting techniques, or by quantitative RT-PCR. RNAi-mediated degradation of target mRNA by an siRNA containing a given target sequence can also be evaluated with animal models, such as mouse models. RNAi-mediated degradation of the target mRNA can be detected by measuring levels of the target mRNA or protein in the cells of a subject, using standard techniques for isolating and quantifying mRNA or protein as described above.

In a preferred embodiment, siRNA molecules target overlapping regions of a desired sense/antisense locus, thereby modulating both the sense and antisense transcripts.

In another preferred embodiment, a composition comprises siRNA molecules, of either one or more, and/or, combinations of siRNAs, siRNAs that overlap a desired target locus, and/or target both sense and antisense (overlapping or otherwise). These molecules can be directed to any target that is desired for potential therapy of any disease or abnormality. Theoretically there is no limit as to which molecule is to be targeted. Furthermore, the technologies taught herein allow for tailoring therapies to each individual.

In preferred embodiments, the oligonucleotides can be tailored to individual therapy, for example, these oligonucleotides can be sequence specific for allelic variants in individuals, the up-regulation or inhibition of a target can be manipulated in varying degrees, such as for example, 10%, 20%, 40%, 100% expression relative to the control. That is, in some patients it may be effective to increase or decrease target gene expression by 10% versus 80% in another patient.

Up-regulation or inhibition of gene expression may be quantified by measuring either the endogenous target RNA or the protein produced by translation of the target RNA. Techniques for quantifying RNA and proteins are well known to one of ordinary skill in the art. In certain preferred embodiments, gene expression is inhibited by at least 10%, preferably by at least 33%, more preferably by at least 50%, and yet more preferably by at least 80%. In particularly preferred embodiments, of the invention gene expression is inhibited by at least 90%, more preferably by at least 95%, or by at least 99% up to 100% within cells in the organism. In certain preferred embodiments, gene expression is up-regulated by at least 10%, preferably by at least 33%, more preferably by at least 50%, and yet more preferably by at least 80%. In particularly preferred embodiments, of the invention gene expression is up-regulated by at least 90%, more preferably by at least 95%, or by at least 99% up to 100% within cells in the organism.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

In a preferred embodiment, small interfering RNA (siRNA) either as RNA itself or as DNA, is delivered to a cell using aptamers. FIG. 2 provides a schematic illustration of aptamer targeted siRNAs.

Many different permutations and combinations of aptamers and RNAi's can be used. For example, the siRNA or oligonucleotide can be attached to one or more aptamers or encoded as a single molecule so that the 5′ to 3′ would encode for an aptamer, the siRNA and an aptamer. These can also be attached via linker molecules. The composition can also comprise in a 5′ to 3′ direction an aptamer attached to another aptamer via a linker which are then attached to the siRNA. These molecules can also be encoded in the same combination. Compositions can include various permutations and combinations. The composition can include siRNAs specific for different polynucleotide targets.

In certain embodiments, the nucleic acid molecules of the present disclosure can be synthesized separately and joined together post-synthetically, for example, by ligation (Moore et al., Science 256:9923, 1992; Draper et al., PCT Publication No. WO 93/23569; Shabarova et al., Nucleic Acids Res. 19:4247, 1991; Belon et al., Nucleosides & Nucleotides 16:951, 1997; Bellon et al., Bioconjugate Chem. 8:204, 1997), or by hybridization following synthesis or deprotection.

In further embodiments. RNAi's can be made as single or multiple transcription products expressed by a polynucleotide vector encoding one or more siRNAs and directing their expression within host cells. An RNAi or analog thereof of this disclosure may be further comprised of a nucleotide, non-nucleotide, or mixed nucleotide/non-nucleotide linker that joins the aptamers and RNAi's. In one embodiment, a nucleotide linker can be a linker of more than about 2 nucleotides length up to about 50 nucleotides in length. In another embodiment, the nucleotide linker can be a nucleic acid aptamer.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991; Arnold et al., PCT Publication No. WO 89/02439; Usman et al., PCT Publication No. WO 95/06731; Dudycz et al., PCT Publication No. WO 95/11910 and Ferentz and Verdine, J. Am. Chem. Soc. 113:4000, 1991.

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In accordance with the invention, siRNA oligonucleotide therapies comprise administered siRNA oligonucleotide which contacts (interacts with) the targeted mRNA from the gene, whereby expression of the gene is modulated. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an siRNA oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the siRNA oligonucleotide.

In another preferred embodiment, the nucleobases in the siRNA may be modified to provided higher specificity and affinity for a target mRNA. For example nucleobases may be substituted with LNA monomers, which can be in contiguous stretches or in different positions. The modified siRNA, preferably has a higher association constant (Kₐ) for the target sequences than the complementary sequence. Binding of the modified or non-modified siRNA's to target sequences can be determined in vitro under a variety of stringency conditions using hybridization assays and as described in the examples which follow.

A fundamental property of oligonucleotides that underlies many of their potential therapeutic applications is their ability to recognize and hybridize specifically to complementary single stranded nucleic acids employing either Watson-Crick hydrogen bonding (A-T′ and G-C) or other hydrogen bonding schemes such as the Hoögsteen/reverse Hoögsteen mode. Affinity and specificity are properties commonly employed to characterize hybridization characteristics of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

The utility of an siRNA oligonucleotide for modulation (including inhibition) of an mRNA can be readily determined by simple testing. Thus, an in vitro or in vivo expression system comprising the targeted mRNA, mutations or fragments thereof, can be contacted with a particular siRNA oligonucleotide (modified or un modified) and levels of expression are compared to a control, that is, using the identical expression system which was not contacted with the siRNA oligonucleotide.

Aptamer-oligonucleotides oligonucleotides may be used in combinations. For instance, a cocktail of several different siRNA modified and/or unmodified oligonucleotides, directed against different regions of the same gene, may be administered simultaneously or separately.

In the practice of the present invention, target gene products may be single-stranded or double-stranded DNA or RNA. Short dsRNA can be used to block transcription if they are of the same sequence as the start site for transcription of a particular gene. See, for example, Janowski et al. Nature Chemical Biology, 2005, 10:1038. It is understood that the target to which the siRNA oligonucleotides of the invention are directed include allelic forms of the targeted gene and the corresponding mRNAs including splice variants. There is substantial guidance in the literature for selecting particular sequences for siRNA oligonucleotides given a knowledge of the sequence of the target polynucleotide. Preferred mRNA targets include the 5′ cap site, tRNA primer binding site, the initiation codon site, the mRNA donor splice site, and the mRNA acceptor splice site.

Where the target polynucleotide comprises an mRNA transcript, sequence complementary oligonucleotides can hybridize to any desired portion of the transcript. Such oligonucleotides are, in principle, effective for inhibiting translation, and capable of inducing the effects described herein. It is hypothesized that translation is most effectively inhibited by the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

Chimeric/modified Molecules: In accordance with this invention, persons of ordinary skill in the art will understand that mRNA includes not only the coding region which carries the information to encode a protein using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. In preferred embodiments, the oligonucleotide is targeted to a translation initiation site (AUG codon) or sequences in the coding region, 5′ untranslated region or 3′-untranslated region of an mRNA. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

Certain preferred oligonucleotides and aptamers of this invention are chimeric. “Chimeric oligonucleotides” or “chimeras,” in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for enzymes capable of cleaving RNA:DNA or RNA:RNA hybrids. By way of example, RNase His a cellular endonuclease which cleaves the RNA strand of an RNA: DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region modified to increase target binding affinity, and, usually, a region that acts as a substrate for RNAse H. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target.

In another preferred embodiment, the region of the oligonucleotide which is modified comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrymidines, abasic residues or an inverted base at the 3′ end of the RNA. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide.

Nuclease resistance is routinely measured by incubating oligonucleotides with cellular extracts or isolated nuclease solutions and measuring the extent of intact oligonucleotide remaining over time, usually by gel electrophoresis, Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃OCH₃, OCH₃O(CH₂), CH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)](Martin et al. Hev. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide, Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (NMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W.H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-5-tritylthiol (Manoharan et al. Ann. N.Y. Acad. Sci. 1992, 660, 306; Manoharan et al. Bioorg. M ed. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimnie 1993, 75, 49), a phospholipid, e.g., di-hexadecyl-rac-glycerol or triethylannmmonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

In accordance with the invention, use of modifications such as the use of LNA monomers to enhance the potency, specificity and duration of action and broaden the routes of administration of oligonucleotides comprised of current chemistries such as MOE, ANA, FANA, PS etc (Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

In a preferred embodiment, siRNA's target genes that prevent the normal expression or, if desired, over expression of genes that are of therapeutic interest as described above. As used herein, the term “overexpressing” when used in reference to the level of a gene expression is intended to mean an increased accumulation of the gene product in the overexpressing cells compared to their levels in counterpart normal cells. Overexpression can be achieved by natural biological phenomenon as well as by specific modifications as is the case with genetically engineered cells. Overexpression also includes the achievement of an increase in cell survival polypeptide by either endogenous or exogenous mechanisms. Overexpression by natural phenomenon can result by, for example, a mutation which increases expression, processing, transport, translation or stability of the RNA as well as mutations which result in increased stability or decreased degradation of the polypeptide. Such examples of increased expression levels are also examples of endogenous mechanisms of overexpression. A specific example of a natural biologic phenomenon which results in overexpression by exogenous mechanisms is the adjacent integration of a retrovirus or transposon. Overexpression by specific modification can be achieved by, for example, the use of siRNA oligonucleotides described herein.

An siRNA polynucleotide may be constructed in a number of different ways provided that it is capable of interfering with the expression of a target protein. The siRNA polynucleotide generally will be substantially identical (although in a complementary orientation) to the target molecule sequence. The minimal identity will typically be greater than about 80%, greater than about 90%, greater than about 95% or about 100% identical.

Moieties: The aptamer-oligonucleotides, in some embodiment, further comprise non-nucleic acid moieties/moieties which may take any number of diverse forms depending on the function desired. For example, modifications introduced in the oligonucleotide backbone of the aptamer-siRNA chimeras: (i) To promote cytoplasmic delivery of the endocytosed aptamer-siRNAs, the aptamer-siRNA ODNs are conjugated to peptides which promote cytoplasmic translocation from endosomes, such the HIV derived tat peptide, a fusogenic peptide from influenza hemagglutinin protein, a 9mer Arg oligopeptide and others. (ii) To increase bioavailability the aptamer-siRNA chimeras are conjugated to cholesterol or polyethylene glycol.

As such, the moieties include natural polymers, synthetic polymers, natural ligands and synthetic ligands, as well as combinations of any and all of the foregoing. When the non-nucleic acid entity or moieties take the form of a natural polymer, suitable members may be modified or unmodified. Natural polymers can be selected from a polypeptide, a protein, a polysaccharide, a fatty acid, and a fatty acid ester as well as any and all combinations of the foregoing.

When the present invention contemplates the use of a synthetic polymer for the non-nucleic acid entity or moieties, homopolymers and heteropolymers may be employed. Such homopolymers and heteropolymers are in many ways preferred when they carry a net negative charge or a net positive charge.

It is significant that the above-described construct of the present invention can be designed to exhibit a further and additional biological activity which is usefully imparted by incorporating at least one or more modified nucleotides, nucleotide analogs, nucleic acid moieties, ligands or a combination of any or all of these. Such biological activity may itself take a number of forms, including nuclease resistance, cell recognition, cell binding, and cellular (cytoplasmic) or nuclear localization.

Ligands or chemical modifications can be attached to the nucleic acid, modified nucleic acid or nucleic acid analogue by modification of the sugar, base and phosphate moieties of the constituent nucleotides (Engelhardt et al., U.S. Pat. No. 5,260,433, fully incorporated herein by reference) or to a non nucleic acid segment of such as polysaccharide, polypeptide and other polymers both natural and synthetic. Modifications of sugar and phosphate moieties can be preferred sites for terminal binding of ligands or chemical modifications and other moieties. Modifications of the base moieties can be utilized for both internal or terminal binding of ligands or chemical modifications and other moieties. Modifications which are non-disruptive for biological function such as specific modifications at the 5 positions of pyrimidines (Ward et al., U.S. Pat. No. 4,711,955, and related divisionals) and the 8 and 7 positions of purines (Engelhardt et al., U.S. Pat. No. 5,241,060 and related divisionals; Stavrianopoulos, U.S. Pat. No. 4,707,440 and related divisionals) may be preferred. The contents of each of the aforementioned U.S. patents and their related divisionals are incorporated herein by reference.

Chemical modification can be limited to a specific segment of the construct such as a tail or a gap, or dispersed throughout the molecule.

Ligands or chemical modifications, being any chemical entity, natural or synthetic, which can be utilized in this invention include macromolecules greater than 20,000 M.W. as well as small molecules less that 20,000 M.W. The ligand or ligands can include both macromolecules and small molecules. Macromolecules which can be utilized include a variety of natural and synthetic polymers including peptides and proteins, nucleic acids, polysaccharides, lipids, synthetic polymers including polyanions, polycations, and mixed polymers. Small molecules include oligopeptides, oligonucleotides, monosaccharides, oligosaccharides and synthetic polymers including polyanions, polycations, lipids and mixed polymers. Small molecules include mononucleotides, oligonucleotides, oligopeptides, oligosaccharides, monosaccharides, lipids, sugars, and other natural and synthetic moieties.

Ligands and chemical modifications provide useful properties for nucleic acid transfer such as 1) cell targeting moieties, 2) moieties which facilitate cellular uptake, 3) moieties specifying intracellular localization, 4) moieties which facilitate incorporation into cellular nucleic acid and 5) moieties which impart nuclease resistance.

In a preferred embodiment, the aptamer-oligonucleotide molecules comprise one or more moieties comprising one or more of: polylysine, polyarginine, Antennapedia-derived peptides, HIV derived tat peptide, a fusogenic peptide from influenza hemagglutinin protein, a 9mer Arg oligopeptide, peptide transporters, peptide transduction domains, intracellular localization domain sequences, or combinations thereof.

Moieties which facilitate cellular uptake include inactivated viruses such as adenovirus (Cristiano et al., 1993, Proc Nat'l Acad Sci USA 90:2122: Curiel et al., 1991, Proc Nat'l Acad Sci USA 88:8850, all of which are incorporated by reference); virus components such as the hemaglutinating protein of influenza virus and a peptide fragment from it, the hemagglutinin HA-2 N-terminal fusogenic peptide (Wagner et al., 1992, Proc Nat'l Acad USA 89:7934, also incorporated herein by reference).

Moieties which specify cellular location include: a) nuclear proteins such as histones; b) nucleic acid species such as the snRNAs U1 and U2 which associate with cytoplasmic proteins and localize in the nucleus (Zieve and Sautereauj, 1990, Biochemistry and Molecular Biology 25; 1); 4) moieties which facilitate incorporation into cellular nucleic acid include: a) proteins which function in integration of nucleic acid into DNA. These include integrase site specific recombinases (Argos et al., 1986, EMBO Journal 5: 433); and b) homologous nucleotide sequences to cellular DNA to promote site specific integration.

Moieties which impart nuclease resistance modifications of constituent nucleotides including addition of halogen atoms groups to the 2′ position of deoxynucleotide sugars.

Ligands or chemical modifications can be introduced into aptamer-oligonucleotide molecules either a) directly by conjugation, b) by enzymatic incorporation of modified nucleoside triphosphates c) by reaction with reactive groups present in constituent nucleotides and d) by incorporation of modified segments. These processes include both chemical and enzymatic methods. Enzymatic methods include primer extension, RNA and DNA ligation, random priming, nick translation, polymerase chain reaction, RNA labeling methods utilizing T7, T3 and 5P6 polymerases, terminal addition by terminal transferase. Chemical methods (described in Kricka, 1995 Nonisotopic Probing, Blotting and Sequencing, Academic Press) include direct attachment of ligands or chemical modifications to activated groups in the nucleic acid such as allylamine, bromo, thio and amino; incorporation of chemically modified nucleotides during chemical synthesis of nucleic acid, chemical end labeling; labeling of nucleic acid with enzymes.

In one preferred embodiment the aptamer-oligonucleotide construct of the present invention carries a net positive charge or a net negative charge. Further, the construct can be neutral or even hydrophobic. It should not be overlooked that the construct may comprise unmodified nucleotides and at least one other member or element selected from one or more nucleotide analogs and non-nucleic acid moieties, or both.

### Generation of Aptamers

Aptamers are high affinity single-stranded nucleic acid ligands which can be isolated from combinatorial libraries through an iterative process of in vitro selection known as SELEX™ (Systemic Evolution of Ligands by EXponential enrichment). Aptamers exhibit specificity and avidity comparable to or exceeding that of antibodies, and can be generated against most targets. Unlike antibodies, aptamers, or in this instance aptamer-oligonucleotides fusions, can be synthesized in a chemical process and hence offer significant advantages in terms of reduced production cost and much simpler regulatory approval process. Also, aptamers-siRNAs are not expected to exhibit significant immunogenicity in vivo.

In preferred embodiments, the siRNA is linked to at least one aptamer which is specific for a desired cell and target molecule. In other embodiments, the RNAi's are combined with two aptamers. For example, FIG. 2. The various permutations and combinations for combining aptamers and RNAi's is limited only by the imagination of the user.

Methods of the present disclosure do not require a priori knowledge of the nucleotide sequence of every possible gene variant (including mRNA splice variants) targeted by the RNAi or analog thereof.

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

The SELEX™ process is a method for the in vitro evolution of nucleic acid molecules with highly specific binding to target molecules and is described in, e.g., U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotides comprising randomized sequences derived from chemical synthesis on a standard DNA synthesizer. The oligonucleotides can be modified or unmodified DNA, RNA or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a pre-selected purpose such as, CpG motifs, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nuc. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be either RNA or DNA. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process. In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method can be modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-cross linking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. SELEX™ can also be used to obtain nucleic acid ligands that bind to more than one site on the target molecule, and to obtain nucleic acid ligands that include non-nucleic acid species that bind to specific sites on the target.

Counter-SELEX™ is a method for improving the specificity of nucleic acid ligands to a target molecule by eliminating nucleic acid ligand sequences with cross-reactivity to one or more non-target molecules, Counter-SELEX is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonuclease before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. For example, oligonucleotides containing nucleotide derivatives chemically modified at the 2′ position of ribose, 5 position of pyrimidines, and 8 position of purines, 2′-modified pyrimidines, nucleotides modified with 2′-amino (2′-NH₂), 2′-fluoro (2′-F), and/or 2′-O-methyl (2′-OMe) substituents.

In preferred embodiments, one or more modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH2-CH2-), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. Post-SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method can also be used in embodiments of the invention. Small peptides have flexible structures and usually exist in solution in an equilibrium of multiple conformers.

The aptamers with specificity and binding affinity to the target(s) of the present invention are typically selected by the SELEX™ process as described herein. As part of the SELEX™ process, the sequences selected to bind to the target can then optionally be minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

The results show that the aptamer-RNAi compositions enter cells and sub-cellular compartments. However, further aptamers can be obtained using various methods. In a preferred embodiment, a variation of the SELEX™ process is used to discover aptamers that are able to enter cells or the sub-cellular compartments within cells. These delivery aptamers will allow or increase the propensity of an oligonucleotide to enter or be taken up by a cell. The method comprises the ability to selectively amplify aptamers that have been exposed to the interior of a cell and became modified in some fashion as a result of that exposure. Such modifications include functioning as a template for template-dependent polymerization. This variation of SELEX™ permits the discovery of aptamers that are: (i) completely specific with regard to the kind of cell or sub-cellular compartment, such as the nucleus or cytoplasm, that they permit entry to, (ii) completely generic, or (iii) partially specific.

One potential strategy is to substitute cell-association for cell entry, and after incubation of the library with the cells and subsequent washing of the cells, amplify the library members that remain associated with the cells. However, this may not distinguish between aptamers that permit genuine cell entry and other trivial solutions to the cell-association problem such as binding to the exterior of the cell membrane, entering, but not leaving, the cell membrane and being taken up by, but not leaving, the endosome.

An alternative strategy is to select for some kind of transformation of the oligonucleotide library member that could happen only in the cytoplasm or other sub-cellular compartment, optionally because the library member is conjugated to a transformable entity, and then selectively amplifying the transformed library members. Such markers include, but are not limited to: reverse transcription, RNaseH, kinase, 5′-phosphorylation, 5′-dephosphorylation, translation-dependent, post-transcriptional modification to give restrictable cDNA, transcription-based, ubiquitination, ultracentrifugation, or utilizing the endogenous protein kinase Clp1. For example, library members can have a designed hairpin structure at their 3′-terminus that will reverse-transcribe without a primer. Reverse transcriptase activity is introduced into the cytoplasm using a protein expression vector or virus. The selective amplification of reverse-transcribed sequences is achieved by using a nucleotide composition that will not amplify directly by, for example, PCR such as completely or partially 2′-OH or 2′OMe RNA and omitting an RT step from the procedure.

Identification of Target Nucleic acid Sequences

With an emerging functional RNA world, there are new potential targets to be considered. Among these are large numbers of natural occurring antisense transcripts with a capacity to regulate the expression of sense transcripts including those that encode for conventional drug targets.

In a preferred embodiment, the compositions of the invention target desired nucleic acid sequences. Any desired target nucleic acid sequences can be identified by a variety of methods such as SAGE. SAGE is based on several principles. First, a short nucleotide sequence tag (9 to b.p.) contains sufficient information content to uniquely identify a transcript provided it is isolated from a defined position within the transcript. For example, a sequence as short as 9 b.p. can distinguish 262,144 transcripts given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 b.p. may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags are used to uniquely identify gene products. This is due to their length, and their specific location (3′) in a gene from which they are drawn. The full length gene products can be identified by matching the tag to a gene data base member, or by using the tag sequences as probes to physically isolate previously unidentified gene products from cDNA libraries. The methods by which gene products are isolated from libraries using DINA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above methods but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), READS (restriction enzyme amplification of digested cDNAs) (Prashar and Weissman, Methods Enznmol., 1999, 303, 258-72), TOGA (total gene expression analysis) (Sutcliffe, et al., Proc. Natl. Acad. Sci. U.S.A., 2000, 97, 1976-81), protein arrays and proteonmics (Celis, et al., FEBS Lett., 2000, 480, 2-16; Jungblut, et al., Electrophoresis, 1999, 20, 2100-10), subtractive RNA fingerprinting (SuRF) (Fuchs, et al., Anal. Biochemi., 2000, 286, 91-98; Larson, et al., Cytometry, 2000, 41, 203-208), subtractive cloning, differential display (DD) (Jurecic and Belmont, Curr. Opin. M licrobiol., 2000, 3, 316-21), comparative genomic hybridization (Carulli, et al., J Cell Biochem. Suppl., 1998, 31, 286-96), FISH (fluorescent in situ hybridization) techniques (Going and Gusterson, Eur. Cancer, 1999, 35, 1895-904) and mass spectrometry methods (reviewed in (Comb. Chem. High Throughput Screen, 2000, 3, 235-41)).

In yet another aspect, siRNA oligonucleotides that selectively bind to variants of target gene expression products. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/iBLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In accordance with the invention, paralogs can be identified for designing the appropriate siRNA oligonucleotide. Paralogs are genes within a species that occur due to gene duplication, but have evolved new functions, and are also referred to as isotypes.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) and references cited therein. Alternatively, one of skill in the art can use the identified sequences and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Another suitable method for identifying targets for the aptamer-RNAi compositions includes contacting a test sample with a cell expressing a receptor or gene thereof, an allele or fragment thereof; and detecting interaction of the test sample with the gene, an allele or fragment thereof, or expression product of the gene, an allele or fragment thereof. The desired gene, an allele or fragment thereof or expression product of the gene, an allele or fragment thereof suitably can be detectably labeled e.g. with a fluorescent or radioactive component.

In another preferred embodiment, a cell from a patient is isolated and contacted with a drug molecule that modulates an immune response. The genes, expression products thereof, are monitored to identify which genes or expression products are regulated by the drug. Interference RNA's can then be synthesized to regulate the identified genes, expression products that are regulated by the drug and thus, provide therapeutic oligonucleotides. These can be tailored to individual patients, which is advantageous as different patients do not effectively respond to the same drugs equally. Thus, the oligonucleotides would provide a cheaper and individualized treatment than conventional drug treatments.

In one aspect, hybridization with oligonucleotide probes that are capable of detecting polynucleotide sequences, including genomic sequences, encoding desired genes or closely related molecules may be used to identify target nucleic acid sequences. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding genes, allelic variants, or related sequences.

Probes may also be used for the detection of related sequences, and should preferably have at least 50% sequence identity or homology to any of the identified genes encoding sequences, more preferably at least about 60, 70, 75, 80, 85, 90 or 95 percent sequence identity to any of the identified gene encoding sequences (sequence identity determinations discussed above, including use of BLAST program). The hybridization probes of the subject invention may be DNA or RNA and may be derived from the sequences of the invention or from genomic sequences including promoters, enhancers, and introns of the gene.

“Homologous,” as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ ATTGCC 5′ and 3′ TTTCCG 5′ share 50% homology.

Means for producing specific hybridization probes for polynucleotides encoding target genes include the cloning of polynucleotide sequences encoding target genes or derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³²S, or by enzymatic labels, such as alkaline phosphatase coupled to the probe via avidin-biotin coupling systems, fluorescent labeling, and the like.

The polynucleotide sequences encoding a target gene may be used in Southern or Northern analysis, dot blot, or other membrane-based technologies; in PCR technologies; in dipstick, pin, and multiformat ELISA-like assays; and in microarrays utilizing fluids or tissues from patients to detect altered target gene expression. Gel-based mobility-shift analyses may be employed. Other suitable qualitative or quantitative methods are well known in the art.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding a desired gene expression product may involve the use of PCR. These oligomers may be chemically synthesized, generated enzymatically, or produced in vitro. Oligomers will preferably contain a fragment of a polynucleotide encoding the expression products, or a fragment of a polynucleotide complementary to the polynucleotides, and will be employed under optimized conditions for identification of a specific gene. Oligomers may also be employed under less stringent conditions for detection or quantitation of closely-related DNA or RNA sequences.

In further embodiments, oligonucleotides or longer fragments derived from any of the polynucleotide sequences, may be used as targets in a microarray. The microarray can be used to monitor the identity and/or expression level of large numbers of genes and gene transcripts simultaneously to identify genes with which target genes or its product interacts and/or to assess the efficacy of candidate aptamer-RNAi compositions in regulating expression products of genes that mediate, for example, tumor specific immune responses. This information may be used to determine gene function, and to develop and monitor the activities of compositions.

Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Nat. Acad. Sci. U.S.A. 94: 2150-2155; and Heller et al., 1997, U.S. Pat. No. 5,605,662).

In other preferred embodiments, high throughput screening (HTS) can be used to measure the effects of RNAi's on complex molecular events such as signal transduction pathways, as well as cell functions including, but not limited to, cell function, apoptosis, cell division, cell adhesion, locomotion, exocytosis, and cell-cell communication. Multicolor fluorescence permits multiple targets and cell processes to be assayed in a single screen. Cross-correlation of cellular responses will yield a wealth of information required for target validation and lead optimization.

In another aspect, the present invention provides a method for analyzing cells comprising providing an array of locations which contain multiple cells wherein the cells contain one or more fluorescent reporter molecules; scanning multiple cells in each of the locations containing cells to obtain fluorescent signals from the fluorescent reporter molecule in the cells; converting the fluorescent signals into digital data; and utilizing the digital data to determine the distribution, environment or activity of the fluorescent reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles, Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. (hem. 272:27497-27500; Southwick et al., (1990). Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. M echanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to an RNAi or probes of interest.

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually,

### Assessing Up-Regulation or Inhibition of Gene Expression

Transfer of an exogenous nucleic acid into a host cell or organism can be assessed by directly detecting the presence of the nucleic acid in the cell or organism. Such detection can be achieved by several methods well known in the art. For example, the presence of the exogenous nucleic acid can be detected by Southern blot or by a polymerase chain reaction (PCR) technique using primers that specifically amplify nucleotide sequences associated with the nucleic acid. Expression of the exogenous nucleic acids can also be measured using conventional methods. For instance, mRNA produced from an exogenous nucleic acid can be detected and quantified using a Northern blot and reverse transcription PCR (RT-PCR).

Expression of an RNA from the exogenous nucleic acid can also be detected by measuring an enzymatic activity or a reporter protein activity. For example, siRNA activity can be measured indirectly as a decrease or increase in target nucleic acid expression as an indication that the exogenous nucleic acid is producing the effector RNA. Based on sequence conservation, primers can be designed and used to amplify coding regions of the target genes. Initially, the most highly expressed coding region from each gene can be used to build a model control gene, although any coding or non coding region can be used. Each control gene is assembled by inserting each coding region between a reporter coding region and its poly(A) signal. These plasmids would produce an mRNA with a reporter gene in the upstream portion of the gene and a potential RNAi target in the 3′ non-coding region. The effectiveness of individual RNAi's would be assayed by modulation of the reporter gene. Reporter genes useful in the methods of the present invention include acetohydroxy acid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Methods to determine modulation of a reporter gene are well known in the art, and include, but are not limited to, fluorometric methods (e.g. fluorescence spectroscopy, Fluorescence Activated Cell Sorting (FACS), fluorescence microscopy), antibiotic resistance determination.

Although biogenomic information and model genes are invaluable for high-throughput screening of potential RNAi's, interference activity against target nucleic acids ultimately must be established experimentally in cells which express the target nucleic acid. To determine the interference capability of the RNAi sequence, the RNAi containing vector is transfected into appropriate cell lines which express that target nucleic acid. Each selected RNAi construct is tested for its ability to modulate steady-state mRNA of the target nucleic acid. In addition, any target mRNAs that “survive” the first round of testing are amplified by reverse transcriptase-PCR and sequenced (see, for example, Sambrook, J. et al. “Molecular Cloning: A Laboratory Manual,” 2nd addition, Cold Spring Harbor Laboratory Press, Plainview, N.Y. (1989)). These sequences are analyzed to determine individual polymorphisms that allow mRNA to escape the current library of RNAi's. This information is used to further modify RNAi constructs to also target rarer polymorphisms.

Methods by which to transfect cells with RNAi vectors are well known in the art and include, but are not limited to, electroporation, particle bombardment, mnicroinjection, transfection with viral vectors, transfection with retrovirus-based vectors, and liposome-mediated transfection. Any of the types of nucleic acids that mediate RNA interference can be synthesized in vitro using a variety of methods well known in the art and inserted directly into a cell. In addition, dsRNA and other molecules that mediate RNA interference are available from commercial vendors, such as Ribopharma AG (Kulmach, Germany), Eurogentec (Seraing, Belgium), Sequitur (Natick, Mass.) and Invitrogen (Carlsbad, Calif.). Eurogentec offers dsRNA that has been labeled with fluorophores (e.g., HEX/TET; 5′-Fluorescein, 6-FAM; 3′-Fluorescein, 6-FAM; Fluorescein dT internal; 5′ TAMRA, Rhodamine; 3′ TAMRA, Rhodamine), which can also be used in the invention. RNAi molecules can be made through the well-known technique of solid-phase synthesis. Equipment for such synthesis is sold by several vendors including, for example, Applied Biosystems (Foster City, Calif.). Other methods for such synthesis that are known in the art cart additionally or alternatively be employed. It is well-known to use similar techniques to prepare oligonucleotides such as the phosphorothioates and alkylated derivatives.

RNA directly inserted into a cell can include modifications to either the phosphate-sugar backbone or the nucleoside. For example, the phosphodiester linkages of natural RNA can be modified to include at least one of a nitrogen or sulfur heteroatom. The interfering RNA can be produced enzymatically or by partial/total organic synthesis. The constructs can be synthesized by a cellular RNA polymerase or a bacteriophage RNA polymerase (e.g., T3, T7, SP6). If synthesized chemically or by in vitro enzymatic synthesis, the RNA can be purified prior to introduction into a cell or animal. For example, RNA can be purified from a mixture by extraction with a solvent or resin, precipitation, electrophoresis, chromatography or a combination thereof as known in the art. Alternatively, the interfering RNA construct can be used without, or with a minimum of purification to avoid losses due to sample processing. The RNAi construct can be dried for storage or dissolved in an aqueous solution. The solution can contain buffers or salts to promote annealing, and/or stabilization of the duplex strands. Examples of buffers or salts that can be used in the present invention include, but are not limited to, saline, PBS, N-(2-Hydroxyethyl)piperazin-e-N′-(2-ethanesulfonic acid) (HEPES™), 3-(N-Morpholino)propanesulfonic acid (MOPS), 2-bis(2-Hydroxyethylene)amino-2-(hydroxymethyl)-1,3-propaned-iol (bis-TRIS™), potassium phosphate (KP), sodium phosphate (NaP), dibasic sodium phosphate (Na₂HPO₄), monobasic sodium phosphate (NaH₂PO₄), monobasic sodium potassium phosphate (NaKHPO₄), magnesium phosphate (Mg₃ (PO₄)₂-4-H₂O), potassium acetate (CH3COOH), D(+)-α-sodium glycerophosphate (HOCH₂CH(OH)CH₂OPO₃Na₂) and other physiologic buffers known to those skilled in the art. Additional buffers for use in the invention include, a salt M-X dissolved in aqueous solution, association, or dissociation products thereof, where M is an alkali metal (e.g., Li⁺, Na⁺, K⁺, Rb⁺), suitably sodium or potassium, and where X is an anion selected from the group consisting of phosphate, acetate, bicarbonate, sulfate, pyruvate, and an organic monophosphate ester, glucose 6-phosphate or DL-α-glycerol phosphate.

### Pharmaceutical Compositions

The invention also includes pharmaceutical compositions containing nucleic acid conjugates. In some embodiments, the compositions are suitable for internal use and include an effective amount of a pharmacologically active conjugate of the invention, alone or in combination, with one or more pharmaceutically acceptable carriers. The conjugates are especially useful in that they have very low, if any toxicity.

Compositions of the invention can be used to treat, prevent, diagnose or image a pathology, such as a disease or disorder, or alleviate the symptoms of such disease or disorder in a patient. For example, compositions of the invention can be used to treat, prevent, diagnose or image a pathology associated with inflammation. Compositions of the invention are useful for administration to a subject suffering from, or predisposed to, a disease or disorder which is related to or derived from a target to which the aptamers specifically bind or to the polynucleotides which the aptamer-delivered RNAi's are targeted to.

Compositions of the invention can be used in a method for treating a patient having a pathology, e.g. cancer. The method involves administering to the patient a composition comprising aptamers-RNAi's that bind a target (e.g., a protein), so that the RNAi is specifically delivered to a target cell of choice and altering the biological function of the target, thereby treating the pathology.

The patient having a pathology, e.g. the patient treated by the methods of this invention can be a mammal, or more particularly, a human.

In practice, the conjugate or multi-domain molecules (e.g., aptamer-RNAi's), are administered in amounts which will be sufficient to exert their desired biological activity.

Compositions of the invention can be used in a method for inducing or enhancing immunogenicity of a target cell in vitro or in vivo and modulating an immune response in patient comprising: obtaining a composition comprising at least one aptamer conjugated to at least one oligonucleotide molecule wherein the aptamer is specific for a desired target cell and the oligonucleotide is specific for a molecule associated with at least one factor associated with a nonsense mediated decay pathway (NMD); and, administering the composition in a therapeutically effective amount to the patient. Examples of target cells comprise: a tumor cell, an infected cell, a tissue specific cell, an adipocyte, a stem cell, an immune cell, an organ specific cell or a transformed cell.

In another preferred embodiment, an antigen specific immune cell is optionally co-stimulated comprising administering to a patient co-stimulatory inducing agent is optionally administered to the patient. In preferred embodiments, the immune cells are specific for the novel antigens induced by the modulation of the NMD pathways, as well as any other antigen expressed by an abnormal cell, for example, PSMA.

In a preferred embodiment, an immune cell co-stimulatory agent targets one or more molecules comprising: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), INFRSF 4, ATAR, LIGHTR, TR2), CD150 (SLAM), CD152 (CTLA-4), CD54, (CD400L), CD178 (F asL), CD209 (DC-SIGN), CD 270, CD277, AITR, AITRL, B7-H3, B7-14, BTLA, HLA-ABC, HLA-IDR, ICOS, ICOSL (B7RP-1), NKG2D), PD-1 (CD279), PD-L1 (B7-H), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ) T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

In another preferred embodiment, immune cells comprise T cells (T lymphocytes), B cells (B lymphocytes), antigen presenting cells, dendritic cells, monocytes, macrophages, myeloid suppressor cells, natural killer (NK) cells, NK T cells, suppressor cells, T regulatory cells (Tregs), cytotoxic T lymphocytes (CTLs), CTL lines, CTL clones, CTLs from tumor, inflammatory, or other infiltrates and subsets thereof.

One aspect of the invention comprises a pharmaceutical composition of the invention in combination with other treatments for inflammatory and autoimmune diseases, cancer, and other related disorders. The pharmaceutical compositions of the invention may contain, for example, more than one aptamer-RNAi. In some examples, a pharmaceutical composition of the invention, containing one or more compounds of the invention, is administered in combination with another useful composition such as an anti-inflammatory agent, an immunostimulator, a chemotherapeutic agent, an antiviral agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of an aptamer-RNAi conjugate of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. Combination therapy also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

For any aptamer-RNAi used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the peptides described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Dosage amount and interval may be adjusted individually to provide plasma levels of the active moiety which are sufficient to maintain therapeutic effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferable between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or other known methods.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The compositions of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compositions of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compositions for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with art aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate. The dosage regimen utilizing the aptamer-RNAi's is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoneal dosages will range between 0.05 to 3800 mg/day. Effective plasma levels of the compounds of the present invention range from 0.002 mg/mL to 50 mg/mL. Compounds of the present invention may be administered in a single daily dose, or the total daily dosage may be administered in divided doses of two, three or four times daily.

### Other Embodiments

The foregoing paragraphs have described a preferred embodiment in which aptamers, RNAi's and aptamer-RNAi conjugates are synthesized. As those skilled in the art will readily appreciate, RNAi can also be produced through intramolecular hybridization of complementary regions within a single RNA molecule. An expression unit for synthesis of such a molecule comprises the following elements, positioned from left to right: 1. A DNA region comprising a viral enhancer; 2. A DNA region comprising an immediate early or early viral promoter oriented in a 5′ to 3′ direction so that a DNA segment inserted into the region of part 4 is transcribed; 3. A DNA region into which a DNA segment can be inserted. Preferably this region contains at least one restriction enzyme site; 4. A DNA region comprising a transcriptional terminator arranged in a 5′ to 3′ orientation so that a transcript synthesized in a left to right direction from the promoter of part 2 is terminated.

### Kits

In yet another aspect, the invention provides kits for targeting nucleic acid sequences of cells and molecules associated with modulation of the immune response in the treatment of diseases such as, for example, infectious disease organisms, cancer, autoimmune diseases and the like. For example, the kits can be used to target any desired nucleic sequence and as such, have many applications.

In one embodiment, a kit comprises: (a) an aptamer-RNAi that targets a desired cell and nucleic acid sequence, and (b) instructions to administer to cells or an individual a therapeutically effective amount of aptamer-RNAi. In some embodiments, the kit may comprise pharmaceutically acceptable salts or solutions for administering the aptamer-RNAi. Optionally, the kit can further comprise instructions for suitable operational parameters in the form of a label or a separate insert. For example, the kit may have standard instructions informing a physician or laboratory technician to prepare a dose of aptamer-RNAi.

Optionally, the kit may further comprise a standard or control information so that a patient sample can be compared with the control information standard to determine if the test amount of an aptamer-RNAi is a therapeutic amount consistent with for example, a shrinking of a tumor or decrease in viral load in a patient.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Embodiments of the invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### Example 1

### Aptamer Targeted Inhibition of Nonsense Mediated Decay (NMD)

Experimental strategy. The goal of this project is determine whether and to what extent targeted inhibition of NMD in tumor cells is capable of potentiating tumor immunity and inhibition of tumor growth in mice.

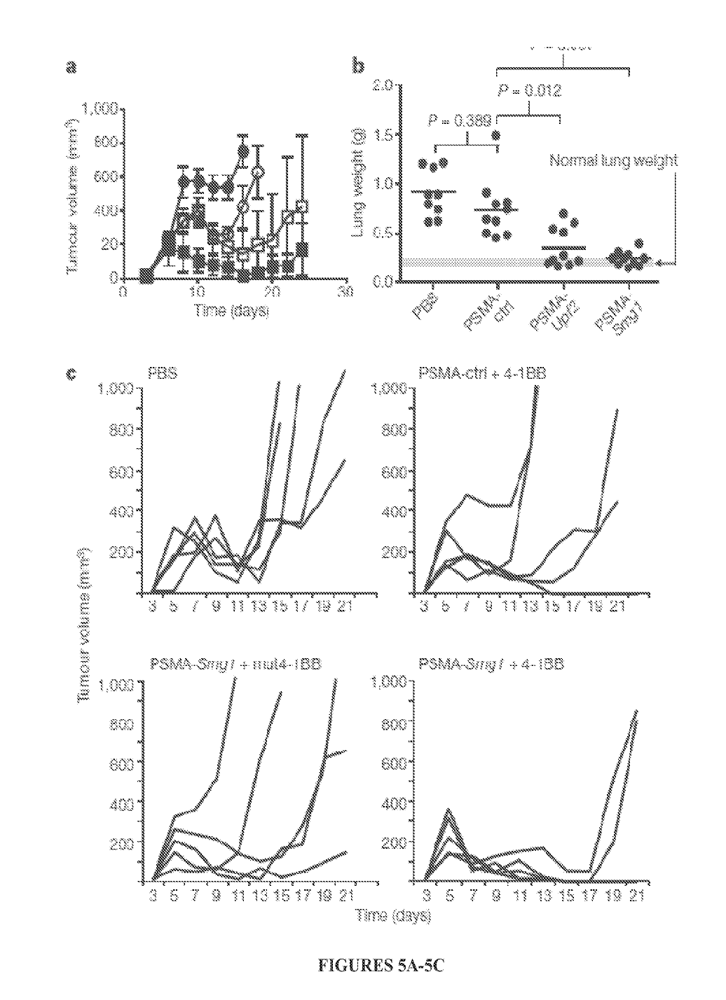

NMD inhibition-induced expression of novel antigens abrogated tumor growth. Given that in this experiment NMD was inhibited in all cells from day zero, from a clinical standpoint—involving targeted delivery of siRNA to tumor bearing individuals—the questions were whether expression of new antigens in an established tumor would suffice to inhibit tumor growth, and what proportion of tumor cells need to express new antigens to exert a therapeutic impact. Expression of novel antigenic determinants in a proportion, e.g. 5-15%, of the tumor cells would be sufficient to stimulate a local immune response through the recruitment of the innate arm of the immune response to eradicate the rest of the tumor. As shown in FIGS. 5 and 6, aptamers can be used to target siRNA to specific cells in vitro and in vivo.

Design of apamer-oligonucleotides. The first objective is to construct aptamer-oligonucleotides fusion ODNs that target the siRNA to tumor cells leading to effective inhibition of NMD.

Aptamers and siRNAs. Aptamers are directed to human PSMA and rat Her2/Neu. Murine tumors, B16 melanoma, 4T1 breast carcinoma, CT26 colon carcinoma, are stably transfected with PSMA; TUBO cell line and spontaneously arising tumors in Balb-NeuT mice, a transgenic model for breast cancer, express rat Her2/neu. siRNAs are generated against murine SMG1, Upf1, Upf2 and Upf3. Aptamer-oligonucleotides fusions are generated using existing algorithms and exploring novel algorithms to maximize the function of the conjugated siRNA. Two modifications are introduced in the oligonucleotide backbone of the aptamer-oligonucleotides chimeras: (i) To promote cytoplasmic delivery of the endocytosed aptamer-oligonucleotides, the aptamer-oligonucleotides ODNs are conjugated to peptides which promote cytoplasmic translocation from endosomes, such the HIV derived tat peptide, a fusogenic peptide from influenza hemagglutinin protein, a 9mer Arg oligopeptide and others. (ii). To increase bioavailability the aptamer-oligonucleotides chimeras are conjugated to cholesterol or polyethylene glycol.

Measuring NMD inhibition. Aptamer-oligonucleotides are first screened for NMD inhibition using a standard assay based on rescuing the stable expression of mRNA from an NMD reporter plasmid encoding a globin transcript with an engineered PTC that accumulates reduced levels of mRNA in transiently transfected cells. Promising aptamer-oligonucleotides are subjected to transcript expression profiling to determine their ability to upregulate multiple NMD substrates. In view of the physiological roles of NMD and other roles of SMG1 and Upf1 discussed above, effects on cell viability and proliferation are closely monitored.

Induction of protective tumor immunity in mice. Effective aptamer-oligonucleotides are tested in murine tumor models for targeted inhibition of NMD in tumor cells and induction of protective immunity.

To optimize the delivery of aptamer-oligonucleotides ODNs, pharmacokinetics studies are first carried out to determine the half-life and biodistribution of the ODNs as a function of dose, frequency and route of administration. Specificity of in vivo tumor targeting expressing the cognate receptor are determined and mice are monitored for adverse effects.

To test the hypothesis that targeted inhibition of NMD in vivo leads to presentation of novel antigens which are under NMD control, an in vivo NMD reporter system is to be developed consisting of stably or transiently transfecting PSMA-expressing tumor cell lines with an OVA gene containing a PTC upstream the H-2b-restricted dominant class I and class II epitopes (OVAPTC), and a downstream intron Targeting in vitro and in vivo the OVA PTC encoded PSMA-expressing tumor cells with a PSMA aptamer-oligonucleotides ODNs should stimulate class I and class II presentation that can be detected using OT-I and OT-II T cells, respectively.

The tumor protective effects of administering aptamer-oligonucleotides chimeras are investigated using increasingly stringent tumor models: (i). Experimental metastasis models such as the PSMA transfected 1316 melanoma (FH-2b) and 4T1 breast carcinoma (H-2d), and rat Her21neu-expressing TUBO cells (H-2d). (ii). Balb-NeuT transgenic model for breast cancer which give rise to spontaneous rat Her2/neu tumors. Aptamer-oligonucleotides ODNs are tested alone and in combination with other immune-based treatments including vaccination, depletion of regulatory T cells, and CTLA-4 blockade, and other aptamer-based strategies developed in our program.

Mechanistic studies—is inhibition of tumor growth due to enhanced tumor antigenicity. As discussed above, inhibition of NMD or its factors can have direct cytotoxic effect that could account for an observed tumor inhibition. To rule out a direct cytotoxic effect in vivo, and to demonstrate that tumor inhibition is immune-mediated, aptamer-oligonucleotides mediated tumor inhibition are measured in nude mice and/or in mice depleted of CD4⁺ and CD8⁺ cells with antibodies. To provide direct evidence that enhancement of tumor immunity is mediated by the expression of novel antigenic determinants, and not “immunogenic death,” the aptamer-oligonucleotides treated mice which rejected the tumor are challenged with NMD-inhibited tumors (tumor cells stably transduced with lentivectors expressing siRNA targeted to N MID factors) and tumor growth is compared to that of non-NMD inhibited tumor challenge. Alternatively, in vitro T cell assay are carried out against NMD-inhibited versus non-NMD inhibited tumor cell targets.

Development of human aptamer-oligonucleotides ODNs. Guided by the murine studies, human aptamer-oligonucleotides fusion ODNs are developed and tested in vitro for NMD inhibition and expression of novel NMD-controlled products. PSMA and human Her2 binding aptamers are used for potential treatment of prostate cancer and Her2 positive breast cancer.

### Example 2

### Enhancing Tumor Antigenicity by Targeted Inhibition of Nonsense Mediated mRNA Decay

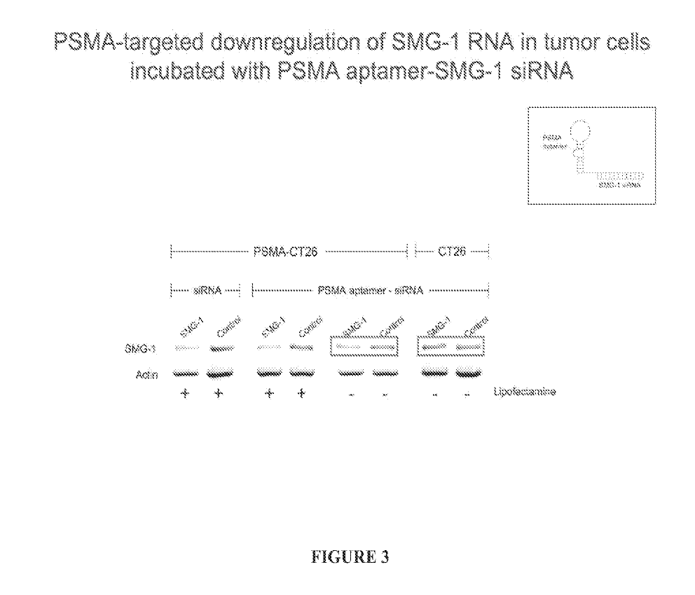

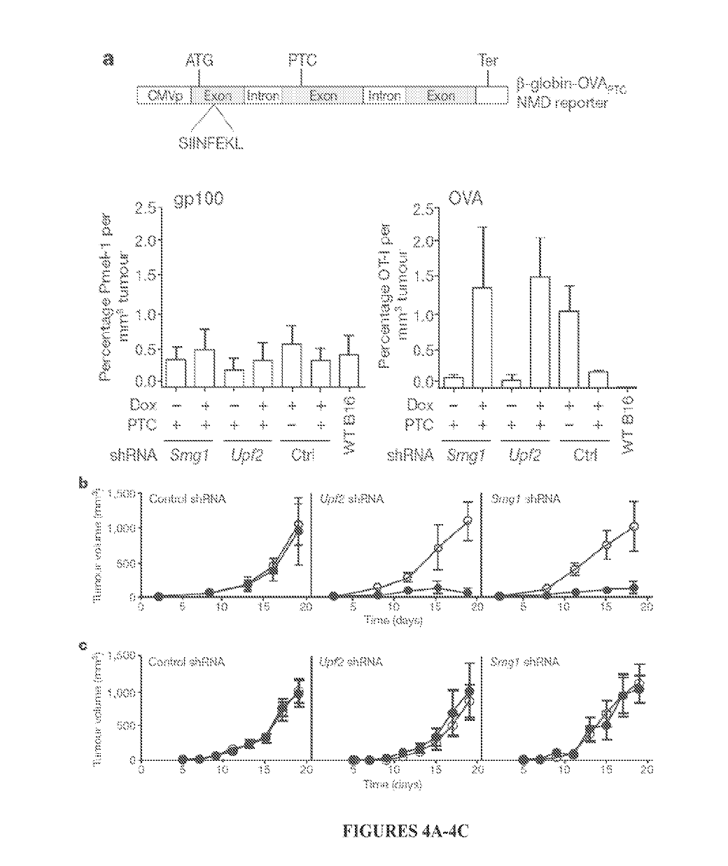

Inhibition of NMD prevents tumor growth: The underlying premise of this approach is that upregulation of gene expression when NMD is inhibited in tumor cells elicits an immune response which leads to tumor rejection. The experiments shown in FIGS. 3 and 4 provide evidence in support of this hypothesis.

Tumor cells stably expressing SMG- and Upf-2 NMD factor siRNAs under doxacycline control were generated by lentiviral transduction. When SMG-1 or Upf-2 siRNAs are expressed (cells are cultured in the presence of drug) the corresponding RNAs are downregulated and NMD is inhibited, NMD inhibition in tumor cells expressing SMG-1 or Upf-2 siRNA (mice receive doxacyline in the drinking water) abrogates tumor growth. Importantly, doxacyline-dependent SMG-1 or Upf-2 siRNA expression has no effect on the exponential growth of tumor cells in culture.

Aptamer targeted delivery of NMD-specific siRNA in vivo inhibits tumor growth: In this experiment, all tumor cells stably expressed siRNA and NMD was inhibited in all tumor cells from day zero. To determine whether inhibition of NMD in a proportion of preexisting tumor cells (determined by the efficiency of the protocol) PSMA aptamer conjugated SMG-1 and Upf-2 siRNAs were used to inhibit NMD in tumor bearing mice.

The experiment depicted in FIG. 3 shows that an SMG-1 siRNA conjugated to a PSMA aptamer is biologically active and is capable of downregulation SMG-1 RNA expression in a PSMA-dependent manner.

To test the robustness of systemically administered PSMA aptamer-SMG-1 or Upf-2 siRNA chimeras to inhibit NMD in tumor bearing mice and reverse tumor growth, the aptamer-oligonucleotides were injected in the tail vein. The results from this experiment shows that systemic administration of PSMA aptamer SMG-1 or Upf-2, but not control, siRNA chimeras to tumor bearing mice inhibits tumor growth. This experiment demonstrates the robustness of the aptamer-oligonucleotides technology and the effectiveness of NMD inhibition to inhibit tumor growth in tumor bearing mice.

Summary: This study provides the outline for a new approach to enhance the antigenicity of disseminated tumor cells. Using a novel oligonucleotide-based platform technology of siRNA conjugated aptamers, NMD was inhibited specifically in tumor cells resulting in tumor regression, conceivably as a result of upregulation of new antigenic products. This approach is clinically feasible, from the standpoint of cost, access to reagents, and regulatory approval process, and broadly applicable to most if not all cancer patients.

### Example 3

### Induction of Tumor Immunity by Targeted Inhibition of Nonsense Mediated mRNA Decay

Methods:

Tumor immunotherapy studies: Three-hundred-thousand parental or pTIG-U6tetOshRNA transduced CT26 tumor cells were implanted subcutaneously in Balb/c or Nude mice. At the day of tumor implantation, mice started receiving water supplemented with 10% sucrose with or without 2 mg ml⁻¹ doxycycline (Sigma).

To evaluate the anti-tumor effects of PSMA aptamer-siRNAs, mice were implanted with 1×10⁶ PSMA-CT26 tumor cells and injected with 400 pmoles of aptamer-siRNA in 100 μl PBS via the tail vein at days 3, 5, 7, 9, 11 and 13. In combination therapy, treatment with PSMA aptamer-siRNA was administered at days 5, 7, 9, 11 and 13, and a single dose of 500 pmoles of 4-1BB aptamer dimer was administered on day 6.

To monitor metastasis, C57BL/6 mice were implanted with 10⁵ B16-PSMA transduced cells by the tail vein and injected with 400 pmoles of aptamer-siRNA conjugates at days 5, 8, 11, 14 and 17. When about half of the mice in the control groups had shown signs of morbidity (approximately days 25-28), the mice were euthanized and their lungs were weighed. GM-CSF-expressing B16/F10 tumor cells were irradiated (50 Gy) and 5×10⁵ cells were injected subcutaneously at days 1, 4 and 7, or days 5, 8 and 11 as described previously (Quezada, S. A., et al. J. Clin. Invest. 116, 1935-1945 (2006)).

For statistical analysis, P values were calculated using a Student's t-test.

PSMA aptamer-siRNA conjugates. The PSMA aptamer, 5′-GGGAGGACGAUGCGGAUCAGCCAUGUUUACGUCACUCCUUGUCAAUCCUCAUCGG CAGACGACACUCGCCCGA-3′ (SEQ ID NO: 1) was cloned into pUC57 between KpnI and BamHI restriction sites. siRNAs were screened using the psiCHECK system (Promega) from candidates generated by the HPCdispatcher and OpenBiosystem algorithms. The DNA template for the aptamer-siRNA guide strand was generated by PCR amplification using forward primer 5′-TAATACGACTCAACTATAGGGAGGIACGATCGG-3′ (SEQ ID NO: 2) and reverse primers 5′-AAGCGTTATGTTTGGTGGAAGTCGGGCGAGTCGTCTG-3′ (SEQ ID NO: 3) for control siRNA, 5′-AAGCCATGACTAACACTGAAATCGGGCGAGTCGTCTG-3′ (SEQ ID NO: 4) Upf2 siRNA, and 5′-AAAATTCTCCGAACGTGTCACTCGGGCGAGTCGTCTG-3′ (SEQ ID NO: 5) for Smg1 siRNA. The PCR products were purified using the QIAprep Spin columns (Qiagen) RNA was transcribed using the T7(Y639F) polymerase and hybridized to the corresponding passenger strands (control siRNA sequence: 5′-AAUUCUCCGAACGUGIUCACdTdT-3′ (SEQ ID NO: 6); Upf2 siRNA sequence: 5′-GCGUUAUGUUUGGUGGAAGdTdT-3 (SEQ ID NO: 7); Smg1 siRNA sequence: 5′-GCCAUCGACUAACACUGAAAdTdT-3′ (SEQ ID NO: 8).

Derivation of PSMA-expressing CT26 tumor cell lines. The PSMA complementary DNA was PCR-amplified using forward primer 5′-GATCAGCGGCCGCCCACCATIGTGGAATCTCCTTCACG-3′ (SEQ ID NO: 9) and reverse primer 5′-GTTAAGTCGACGAGGATCCTCGAGAATCCTCTTAGGCCATTCACTC-3′ (SEQ ID NO: 10), and cloned into the SalI and Not1 restriction sites of the retroviral vector pBMN (Addgene). Plasmid was transiently transfected into the Phoenix-AMPHO 293 packaging cell lines and viral supernatant was used to transduce CT26 colon carcinoma (H-2^{d}) and B16/F10 melanoma (H-2^{b}) tumor cell lines. PSMA-expressing cells were isolated by cell sorting using PSMA-PE labeled anti-PSMA antibody from MBL.

Confocal microscopy. The passenger strand of the siRNAs was labeled with Cy3 before hybridization to the PSMA-aptamer guide strand using the Silencer RNA labeling kit (Ambion). Tumor cells were plated on glass plates, washed with PBS and incubated with 40 nM of Cy3-labelled aptamer-siRNA or with 10 μg ml⁻¹ anti-PSMA antibody (MBL) and Alexa Fluor 488 goat anti-mouse IgG (Molecular Probes). Coverslips were mounted with Prolong Gold-DAPI (Molecular Probes).

Generation of stably transduced shRNA-expressing CT26 and B16/F10 tumor cell lines. Double-stranded oligonucleotides corresponding to the guide and passenger strands of Smg1, Upf2 or control siRNA modified to contain overhangs compatible with BglII and KpnI restriction sites were cloned into the BglIII and KpnI sites of pFRT-U6tetO plasmid. The U6tetO-shRNA cassettes from the pFRT plasmids were isolated by PCR (forward primer: 5′-GATCAGCGGCCGCTGCAGAAGGTCGGGCAGGAAGAG-3′ (SEQ ID NO: 11); reverse primer: 5′-GTTA AGCATGCCCACACTGGACTAGTGGATC-3 (SEQ ID NO: 12) and cloned into the NotI/SphI restriction sites of PTIG lentiviral vector to generate pTIG-U6tetOshRNA plasmids. pTIG-U6tetOshRNA DNA was cotransfected into 293T cells with lentiviral packaging plasmids pCHPG-2, pCMV-rev and PCMV-gag and lentivirus-containing supernatant was collected and concentrated by centrifugation. CT26 colon carcinoma (H-2) and B16/F10 melanoma (H-2^{b}) tumor cell lines were infected with lentiviral vectors and stably transduced GFP-expressing cells were isolated by sorting.

shRNA oligonucleotides used were as follows, Control shRNAs: 5′-GATCAATTCTCCGAACGTGTCACTTCCTGACCCAAAGTGACACGTTCGGAGAATTTT TTTGTAC-3′ (SEQ ID NO: 13); 5∝-AAAAAAATTCTCCGAACGTGTCACTGGGTCAGGAAGTGACACGTTCGGAGAATT-3′ (SEQ ID NO: 14). Up2 shRNA: 5′-GATCGCGTTATGTTTGGTGGAAGAACCTGACCCATTCTTCCACCAAACATAACGCTT TT TTGTAC-3′ (SEQ ID NO: 15); 5′-AAAAAAGCGTTATGTTTGGTGGAAGAATGGGTCAGGTTCTTCTTCACCAAAACATAACG C-3′ (SEQ ID NO: 16). SMg1 shRNA: 59-GATCGCCACCAAAGACATGAGGAAACCTGACCCATTTCCTCATGTCTTTGGTGGCTT TT TGTAC-3′ (SEQ ID NO: 17); 5′-AAAAAGCCACCAAAGACATGAGGAAATGGGTCAGGTTTCCTCATGTCTTTGGTGGC-3′ (SEQ ID NO: 18).

Ct726 and B16/F10 tumor cell lines containing BG, BGPTC and OVA-BGPTC. The SIINFEKL (SEQ ID NO: 19) peptide was cloned into the first exon of the β-globin gene between second (valine) and third (histidine) amino-terminal amino acids of the BG and BGPTC plasmids, by PCR using the forward primer 5′-CCATGGTGAGTATAATAAATTTTGAAAAACTTCACCTGACTCCTGAGGAGAAG-3′ (SEQ ID NO: 20) and reverse primer 5′-GGGTGTTGGCGGGTGTC-3′ (SEQ ID NO: 21), cloned in the pcDNA3.1 plasmid (Invitrogen) and used to transfect parental and pTIG-U6tetOshRNA transduced B16/F10 tumor cells.

RT-PCR. RNA was isolated using RNAsy columns (Qiagen) from cells grown in the presence or absence of 1 μg ml⁻¹ doxycycline (Sigma) for 5 days were reverse-transcribed and PCR-amplified using the following primers. CT26 and pTIG-U6tetOshRNA transduced CT26 tumor cells: actin: forward, 5′-CCACACTGTGCCCATCTACG-3′ (SEQ ID NO: 22); reverse, 5′-GATCTTCATCGGTGCTAGGAGC-3′ (SEQ ID NO: 23). Smg1: forward, 5′-GCCCATCGTGTTTGCTTTGG-3′ (SEQ ID NO: 24); reverse, 5′-TCTCGTTCCCAGTGGTGTTACAG-3′ (SEQ ID NO: 25). Upf2: forward, 5′-ACCCGGGGCUAAUGUIUGAC-3′ (SEQ ID NO: 26); reverse, 5′-CUUGGUAAUGUUAGGCGUUUUCUC-3′ (SEQ ID NO: 27). BG, BGPTC and OVA-BG_{PTC} transduced cells: β-globin: forward, 5′-ACCACCGTAGAACGCAGATCG-3′ (SEQ ID NO: 28); reverse, 5′-CCTGAACTTCTCAGGATCC-3′ (SEQ ID NO: 29).

Transfection of cells with apamer-siRNA confugates. CT26 and PSMA-CT26 tumor cells were incubated with 400 nM siRNA or PSMA aptamer-siRNA conjugate in the presence of absence of Lipofectamine 2000 (Invitrogen) for 2 days and analyzed for RNA expression or NMD inhibition.

Tumor infiltration of OT-1 and Pmel-1 T cells. C57BL/6 mice (CD45.2; Thy 1.2) were implanted subcutaneously with 5×10¹⁶ tumor cells and 8 days after tumor inoculation 5×10⁶ peptide-activated OT-I (CD45.1) or Pmel-1 CD8⁺ cells were injected intravenously via the tail vein. At the same day the drinking water was supplemented with 10% sucrose (Sigma) and with or without 2 mg ml⁻¹ doxycycline (Sigma). At day 14 after tumor implantation mice were euthanized, tumors removed and mechanically disaggregated by collagenase treatment (400 U ml⁻¹). Cells were ficolled and stained with FITC-labeled anti-CD45.1 antibody and allophycocyanin (APC)-labeled anti-CD8 antibody for OT-1 T cells or with phycoerythrin (PE)-labeled anti-Thy1.1 antibody and APC-labeled anti-CD8 antibody for Pmel-I T cells and analyzed by flow cytometry. All antibodies used were from BD Bioscience.

Tumor homing or ³²P-labelled aptamer-siRNA conjugates. The PSMA aptamer was transcribed in vitro in the presence of 1/1,000 parts of α³²P-ATP (3000 Ci mmol⁻¹) (PerkinElmer) and annealed to Smg1 siRNA as described above. Balb/c mice were co-implanted with CT26 and PSMA-CT26 tumor cells in the opposite flanks, and 15 days later injected via the tail vein with 5×10⁵ c.p.m. ³²P-labelled aptamer-siRNA. After aptamer-siRNA injection, tumors were surgically removed, cells dispersed by incubation with 400 U ml⁻¹ of collagenase, washed three times with P3BS, and cell-associated ³²P was measured in a scintillation counter.

Tumor immunotherapy studies. Three-hundred-thousand parental or pTIG-U6tetOshRNA transduced CT26 tumor cells were implanted subcutaneously in Balb/c or Nude mice. At the day of tumor implantation mice started receiving water supplemented with 10% sucrose with or without 2 mg ml⁻¹ doxycycline (Sigma).

To evaluate the anti-tumor effects of PSMA aptamer-siRNAs, mice were implanted with 1×10⁶ PSMA-CT26 tumor cells and injected with 400 pmoles of aptamer-siRNA in 100 ml PBS via the tail vein at days 3, 5, 7, 9, 11 and 13. In combination therapy, treatment with PSMA aptamer-siRNA was administered at days 5, 7, 9, 11 and 13, and a single dose of 500 pmoles of 4-1BB aptamer dimer was administered on day 6.

To monitor metastasis, C57BL/6 mice were implanted with 10⁵ B16-PSMA transduced cells via the tail vein and injected with 400 pmoles of aptamer-siRNA conjugates at days 5, 8, 11, 14 and 17. When about half of the mice in the control groups had shown signs of morbidity (approximately days 25-28), the mice were euthanized and their lungs were weighed. GM-CSF-expressing B16/F10 tumor cells were irradiated (50 Gy) and 5×10⁵ cells were injected subcutaneously at days 1, 4 and 7, or days 5, 8 and 11.

For statistical analysis P values were calculated using a Student's t-test.

Results and Discussion:

Nonsense mediated mRNA decay (NMD), is an evolutionary conserved surveillance mechanism in eukarvotic cells which prevents the expression of mRNAs containing a premature termination codon (PTC) (Behm-Ansmant, I. et al., FEBS Lett. 581, 2845-2853 (2007); Maquat, L. E. Nature Rev. Mol. Cell Biol. 5, 89-99 (2004): Mtihlemann, O. et al., Biochim. Biophys. Acta 1779, 538-549 (2008)). Inhibition of NMD in cultured human cell lines using siRNAs targeted to any of its factors, e.g., SMG1, UPF1, UPF2 or UPF3, results in the upregulation of multiple products encoded by the PTC-containing mRNAs (El-Bchiri, J. et al., PLoS One 3, e2583 (2008); Mendell, J. T. et al., Nature Genet. 36, 1073-1078 (2004); Usuki, F. et al., Mol. Ther. 14, 351-360 (2006); Wittmann, J. et al., Mol. Cell. Biol. 26, 1272-1287 (2006)). Many of such products, resulting from aberrant splicing or NMD dependent autoregulated alternative splicing, encode novel peptides which have not induced tolerance. Without wishing to be bound by theory, it was hypothesized that upregulation of such products when NMD is inhibited in tumor cells would elicit an immune response against (some of) the new products, and that the immune response would inhibit tumor growth. Moreover, there is evidence that frameshift mutations in cancer cells exhibiting DNA mismatch repair (MMR) generate PTC-containing transcripts which are negatively controlled by NMD (Duval, A. & Hamelin, R. Cancer Res. 62, 2447-2454 (2002)). Inhibiting NMD would further augment the production of such tumor-specific antigens.

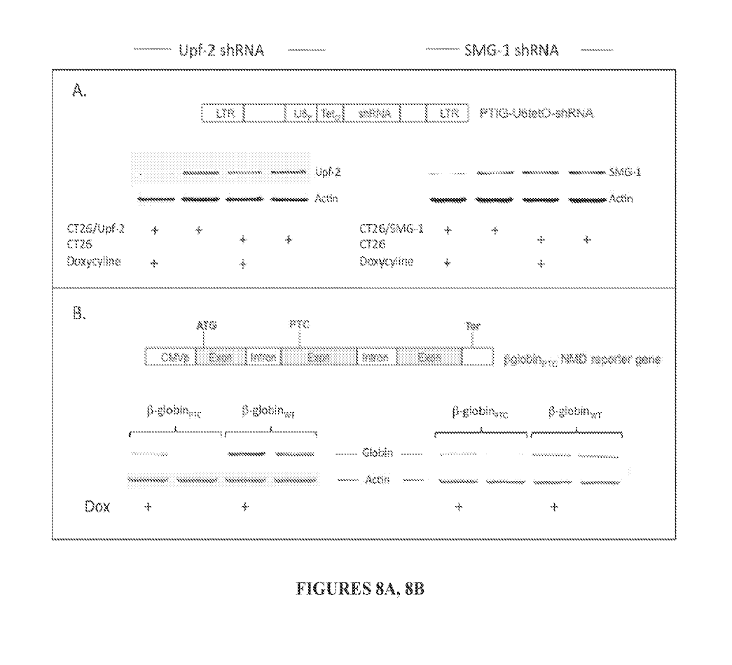

To determine if NMD inhibition in tumor cells can stimulate protective antitumor immunity, it was tested whether stable expression of NMD factor short hairpin RNAs (shRNAs) in tumor cells would inhibit their growth potential in mice. CT26 colon carcinoma tumor cells were transduced with a lentiviral vector (PTIG-U6tetOshRNA) encoding Smg1 or Upf2 shRNAs expressed from a tet-regulated U6 promoter (Aagaard, L. et al. Mol. Ther. 15, 938-945 (2007)). shRNA expression can be upregulated in vitro by adding doxycycline to the culture medium and in vivo by providing doxycycline in the drinking water. Doxycycline-induced Smg1 and Upf2 shRNA expression in cultured CT26 cells results in downregulation of the corresponding mRNA (FIG. 8A) and inhibition of NMD (8FIG. 8B). Long-term inhibition of NMD had no measurable effects on the viability or proliferative capacity of the CT26 cells in vitro.

To determine if siRNA inhibition of NMD in the tumor bearing mice can stimulate immune responses against products which are normally under NMD control, the intratumoral accumulation of T cells was measured, recognizing a model tumor antigen which is suppressed as a result of NMD. B16/F10 tumor cells harboring the doxycycline-inducible Smg1, Upf2, and control shRNAs were stably transfected with an NMD reporter plasmid encoding the dominant MHC class I epitope of the chicken ovalbumin gene (OVA) upstream of a PTC (Diagrams in FIG. 4A and FIG. 8A). Tumor-bearing mice were infused with OT-I transgenic CD8⁺ T cells which recognize the OVA MHC class I-restricted epitope, or with Pmel-1 transgenic CD8⁺ T cells which recognize an MHC class I-restricted epitope in the endogenous gp100 tumor antigen expressed in B16 tumor cells 19, gp100 expression is not under NMD control, As shown in FIG. 4A, unlike Pmel-1 T cells, the OT-I T cells failed to accumulate to significant levels in the OVA negative B16/F10 tumors or in tumors transfected with the PTC-containing β-globin-OVA construct harboring but not expressing Smg1 or Upf2 shRNA. However, upregulation of Smg1 or Upf2, but not control, shRNA (doxycycline in the drinking water), resulted in a significant accumulation of OT-I T cells in the tumors. This experiment showed that siRNA inhibition of NMD in tumor cells induced an immune response in vivo against an antigen which is under NMD control.

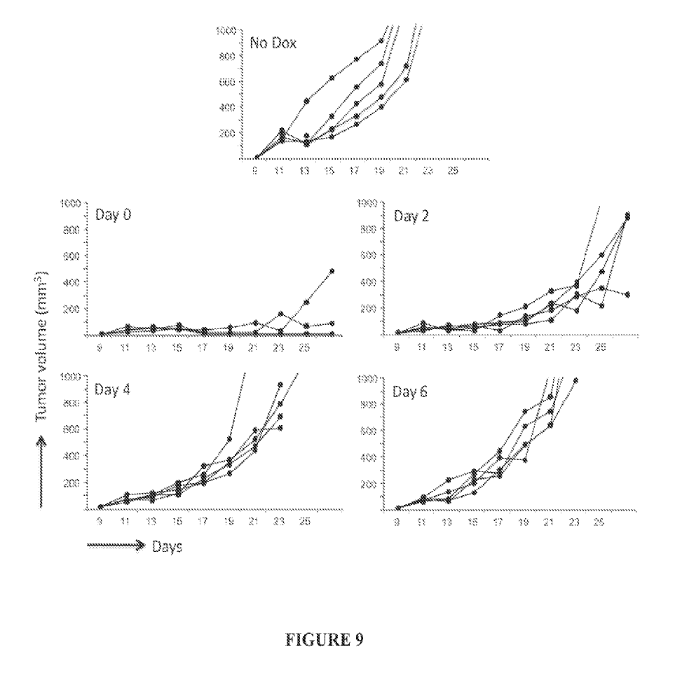

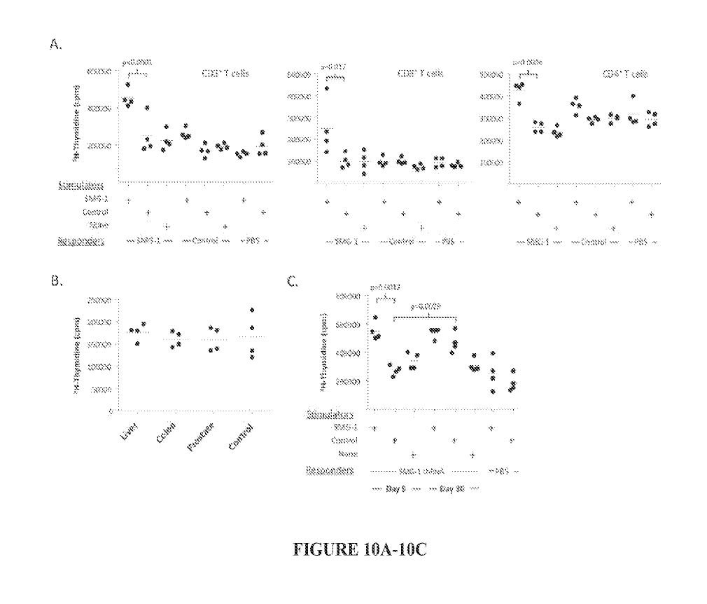

To determine if siRNA-mediated inhibition of NMD affects tumor growth, the lentiviral transduced CT26 cells expressing a control, Smg1 or Upf2 shRNA were implanted subcutaneously into mice and tumor growth was monitored in the presence or absence of doxycycline administered in the drinking water. FIG. 4B shows that tumor cells expressing Smg1 or Upf2, but not control, shRNA grew initially but failed to progress. Tumor inhibition was immune-mediated because the tumors grew in nude mice (FIG. 4C), and mice which rejected the tumors shown in FIG. 4B, but not age-matched control mice, resisted a second challenge with parental tumor cells. Delaying doxycycline treatment of mice expressing SMG-1 shRNA diminished the tumor inhibitory impact which was completely lost when drug treatment was delayed for six days (FIG. 9). Tumor rejection correlated with the induction of T cell responses against tumor cells expressing Smg1 shRNA. No T cell responses were detected against tumor cells which did not express Smg1 or against normal tissues including liver, colon and prostate (FIGS. 10A-10C). This is consistent with the hypothesis that tumor rejection was mediated by the induction of immune responses against NMD-controlled products which were upregulated when NMD was inhibited in the tumor cells.

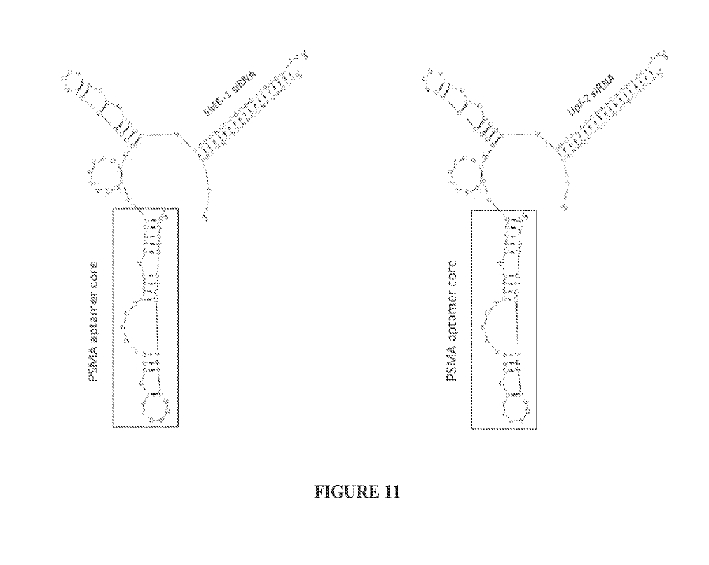

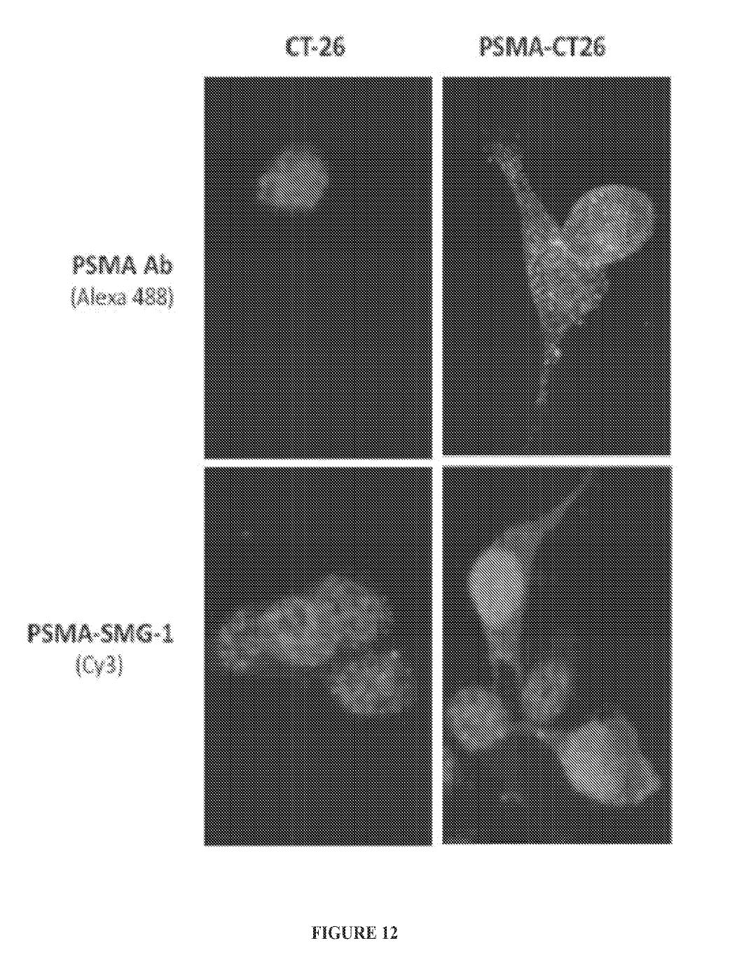

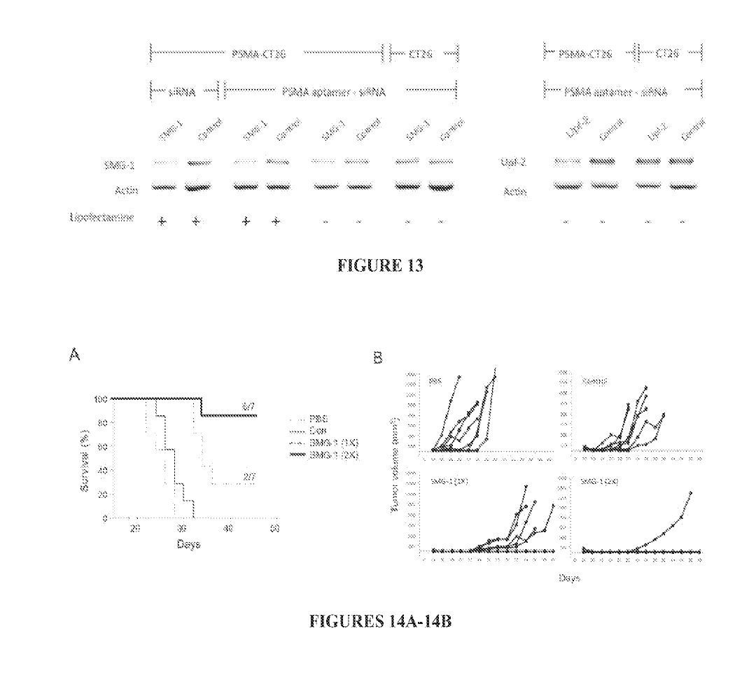

In the experiment shown in FIG. 4B, tumor growth was completely prevented when NMD was inhibited in all tumor cells from the time of tumor implantation. Simulating a more relevant clinical scenario, it was tested whether inhibition of NMD in preexisting tumors can induce therapeutically useful tumor immunity. To preclude NMD inhibition in normal cells, the NMD factor siRNAs were targeted to tumor cells using oligonucleotide aptamer ligands (Gold, L. J. Biol. Chem. 270, 13581-13584 (1995); Nimjee, S. M., et al. Anna. Rev. Med. 56, 555-583 (2005)). Smg1 and Upf2 siRNA were conjugated to an oligonucleotide aptamer which binds to prostate specific membrane antigen (PSMA) as shown in FIG. 11. PSMA expressing CT26 and B16 tumor cell lines were generated by transduction with a PSMA encoding expression vector, and expression of PSMA was confirmed by flow cytometry. The PSMA conjugated siRNAs bound to and were taken up by PSMA-expressing, but not parental, tumor cells (FIG. 12), leading to the downregulation of their target RNAs (FIG. 13).

It was next tested whether systemic administration of PSMA aptamer-siRNA conjugates by tail vein injection can inhibit tumor growth. As shown in FIG. 5A, treatment of day 3 subcutaneously implanted PSMA-CT26 tumor cells with PSMA conjugated Smg1 siRNA, and to a lesser extent Upf2 siRNA, significantly inhibited tumor growth. Two out of seven mice treated with the PSMA aptamer-Smg1 siRNA conjugate rejected the implanted tumors and remained tumor-free (FIG. 14A, FIG. 14B). When treatment intensity was increased by doubling the dose of the aptamer-siRNA conjugate and extending treatment to seven injections, six of the seven mice rejected the tumor long term. Treatment with PSMA aptamer conjugated to control siRNA had a small inhibitory effect which could have resulted from the binding of the PSMA aptamer-siRNA to the tumor cells, or due to nonspecific immune stimulatory effects of the oligonucleotide. No elevated levels of IFNα were found in the serum of mice treated with PSMA aptamer-control, or Smg1 siRNA conjugates. As shown in FIG. 5B, treatment of day five PSMA-B16/F10 tumor implanted mice with PSMA aptamer conjugated Upf2 or Smg1 siRNA inhibited the development of lung metastasis which was more profound in the SMG-1 group. To determine whether NMD inhibition elicited antitumor response can be further enhanced by costimulation, PSMA-CT26 tumor bearing mice were treated with PSMA aptamer-Smg1 siRNA and an agonistic 4-1131BB aptamer dimer (McNamara, J. O. et al. J. Clin. Invest. 118, 376-386 (2008)). The stringency of NMD inhibition and 4-1BB costimulation was adjusted to elicit a limited antitumor effect when applied separately by delaying treatment with PSMA aptamer-siRNA conjugates from day 3 to day 5 and administering a single dose of 4-1BB aptamer on day 6. As shown in FIG. 5C, combination therapy with PSM A aptamer-Smg1 siRNA and 4-1BB aptamer was synergistic.

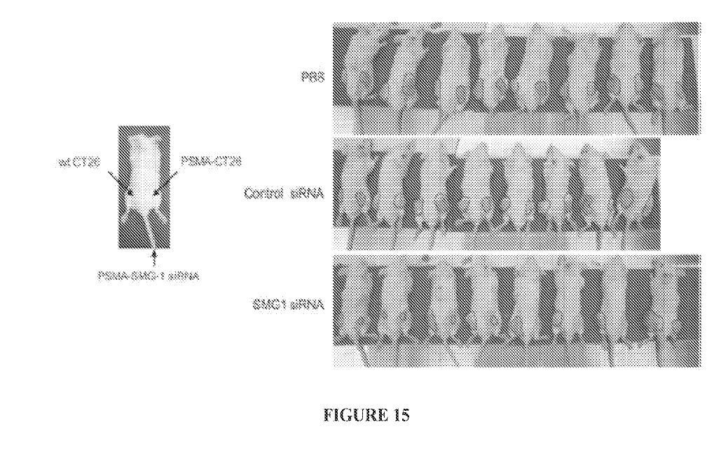

To determine if tumor inhibition shown in FIGS. 5A-5C is a result of aptamer targeting of siRNA to PSMA-expressing tumor cells, mice were implanted in opposite flanks with PSMA-expressing and parental CT26 tumor cells and PSMA aptamer conjugated to control or Smg1 siRNA was administered systemically by tail vein injection (FIG. 6A). FIG. 6B shows that ³²P-labeled PSMA aptamer-Smg1 siRNA conjugate accumulated preferentially in PSMA-expressing tumor cells. FIG. 6C shows that systemic administration of PSMA aptamer conjugated Smg1, but not control, siRNA inhibited the growth of PSMA-expressing CT26 tumor cells but not the contralaterally implanted parental CT26 tumor cells. FIG. 15 shows a snapshot of the tumor-bearing mice at the day of sacrifice.

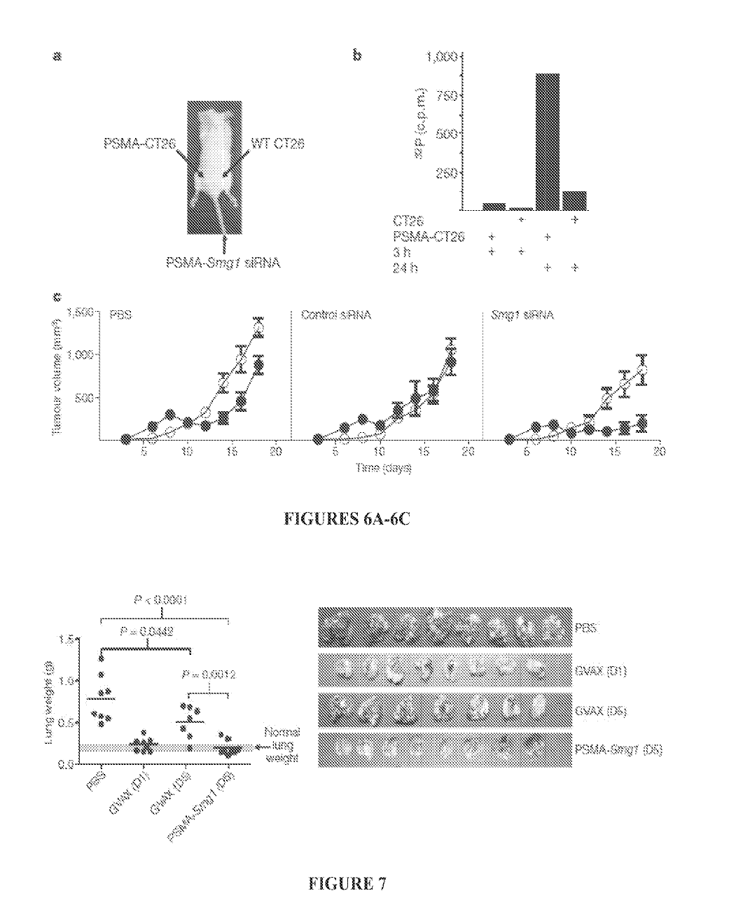

To assess the potency of tumor targeted NMD inhibition, the antitumor effects of treating tumor bearing mice were compared with PSMA aptamer-Smg1 siRNA conjugate and vaccination with GM-CSF-expressing irradiated syngeneic tumor cells (GVAX), a best-in-class tumor vaccination protocol (Jinushi, M., et al. Immunol. Rev. 222, 287-298 (2008); Dranoff, G. et al. Proc. Natl. Acad. Sci. USA 90, 3539-3543 (1993)). In therapeutic protocols when vaccination is initiated 2-4 days post tumor inoculation, the antitumor impact of GVAX is limited, unless combined with other treatments such as CTLA-4 blockade (van Elsas, A., et al. J. Exp. Med. 190, 355-366 (1999)) or T-regulatory cell depletion (Quezada, S. A., et al. J. Clin. Invest. 116, 1935-1945 (2006)). As shown in FIG. 7, in the B16 lung metastasis model described in FIG. 5B, GVAX treatment of day one tumor bearing mice significantly inhibited metastasis whereas treatment of day five tumor bearing mice had a limited antimetastatic effect which barely reached statistical significance. By comparison, treatment of day five tumor bearing mice with PSMA aptamer-Smg1 siRNAs inhibited metastasis to an extent comparable to that of administering GVAX at day one. Given that these are first generation aptamer-siRNA conjugates and the dose and schedule of aptamer-siRNA treatment have not been optimized, these results evidence that tumor targeted siRNA-mediated NMD inhibition is more effective than a best-in-class “conventional” vaccination protocol.

Tumor targeted NMD inhibition is a novel approach to stimulate protective antitumor immunity. Instead of stimulating or potentiating immune responses against existing, often weak, antigens expressed in the tumor cells, the goal of current tumor vaccination protocols, NMD inhibition generates novel antigenic determinants in situ in the disseminated tumor lesions. It should be noted that NMD control of gene expression is “leaky”. In addition to the first round of translation, known as pioneer translation, the efficiency of nonsense-mediated degradation varies among individual mRNA targets. Immune recognition is, therefore, a consequence of upregulation of NMD controlled products above a certain threshold that was set by the natural immune tolerance mechanisms. The NMD inhibition strategy described in this study consists of a single reagent that can be synthesized in a cell-free chemical process; it obviates the need to identify TRAs or adjuvants, and is broadly applicable as it targets a common pathway in all tumors. The potency of the NMD inhibition approach was sevidenced when compared to GVAX vaccination, a “gold standard” best-in-class vaccination protocol. Arguably, this first generation aptamer-siRNA conjugates and the dose and treatment schedule can be further optimized. It would be of interest to determine in future studies whether the NMD-induced antigens are cross-reactive among different tumors, identify the dominant antigens induced by NMD inhibition, and whether “epitope spread” to constitutively expressed tumor antigens contributes to protective immunity.

Physiological roles of NMD—NMD controlled products encode novel peptides. It was initially thought that the main role of NMD was to maintain the proteome integrity of the cell by eliminating transcripts with nonsense mutations generating premature termination codons (PTCs) yielding truncated products. Indeed, over 30% of genetic disorders are caused by PTCs (Frischmeyer, P. A. & Dietz, H. C. Hum Mol Genet. 8, 1893-1900 (1999); Holbrook, J. A., et al. Nat Genet 36, 801-808 (2004)). Truncated products generated by PTCs are not good substrates for generating novel antigenic determinants because the normal expression of the non-truncated products in the absence of a PTC will have triggered tolerance. Yet, nonsense mutations generating PTCs are rare events and it is unlikely that the NMD system has evolved to counter their potential deleterious effects.

It may be that the main and physiological role of the NMD is to regulate normal gene expression. Such products will encode novel peptides and hence could provide antigenic determinants to which the immune system has not be tolerized. For example, an important role of NMD is to maintain splicing integrity. The efficiency and accuracy of splicing is notoriously imperfect. Such transcripts, encoding novel peptides corresponding to intron sequences, will often contain PTCs and hence become targets for NMD elimination (Behm-Ansmant, I. et al. FEBS Lett 581, 2845-2853 (2007); Isken, O. & Maquat, L. E. Nat Rev Genet. 9, 699:712 (2008)). NMD is also responsible for the elimination of transcripts encoding nonproductively rearranged T cell receptors and immunoglobulin chain. A significant proportion of gene products (>15%) that are upregulated when NMD is inhibited, such as by targeting Upf-1 with siRNA, are involved in amino acid biosynthesis and transcription factors which coordinate cellular responses to starvation. Since starvation also downregulates translation thru phosphorylation and inhibition of eIF2α, which in turn inhibits NMD efficiency, it appears that the response to starvation is in part under NMD control. NMD is also implicated in several instances of products autoregulating alternative splicing (e.g., serine-arginine (SR)-rich proteins and hnRNP splicing factors such as SC35, calpain, CDC-like kinases), biosynthesis of selenoproteins, and telomere synthesis (Htolbrook, J. A., et al. Nat Genet. 36, 801-808 (2004); Isken, O. & Maquat, L. E. Nat Rev Genet. 9, 699:712 (2008)). Thus in all such instances, the PTC-containing transcripts will encode novel peptides or consist of regulated gene products that have triggered little or no tolerance.

Role of NMD in cancer, Cancer cells accumulate elevated levels of PTC containing NMD mRNA substrates. About 15% of cancers exhibit defects in DNA mismatch repair (MMR) often manifested as microsatellite instability (MSI). Such defects affecting many products, including products associated with tumor progression such as TGFβRII, APAF-1, IGFIIR, BAX, PTEN, RHAMM, give rise to frameshift mutations resulting in PTCs, Such PTC-containing transcripts are under NMD control whereby Upf-1 siRNA mediated inhibition of NMD in a human colorectal cancer cell line exhibiting an MSI phenotype stabilized the frameshifted mutant transcripts. Such products could provide a source of tumor-specific antigenic determinants downstream of the recombination site. Consistent with this hypothesis, decreased immune infiltrate are seen in tumors with MSI phenotype which correlates with increased levels of Upf-1 in the tumors. Inhibiting NMD will further augment the production of such tumor-specific antigens.

Induction of antiltumor immunity against parental tumor—epitope spread. The mice induced to express SMG-1 or Upf-2 siRNA which rejected the tumors (FIG. 4B) were completely resistant to a subsequent challenge with parental tumor cells (7/7 mice). This is consistent with epitope spread whereby an initial immune response directed to antigens induced by Upf-2 or SMG-1 siRNA inhibition of NMD “spreads” to antigens expressed by the parental tumor. The underlying mechanism of “epitope spread” is that the immune response against the original antigen leads to the destruction of a proportion of the tumor targets, resulting in the release of endogenous antigens which are captured by local professional antigen presenting cells such as dendritic cells and presented to the immune system.

The observation that tumors in which NMD is inhibited elicit immune responses against the parental tumor appears, however, to be inconsistent with the results of FIG. 6C which shows that the PSMA aptamer targeted siRNA inhibition of tumor growth was local; it affected only the PSMA-expressing tumors but not the contralaterally implanted parental tumor cells, Clearly there was no evidence for epitope spread in this instance. A likely explanation that reconciles both observations is that the immune response induced by epitope spread to the endogenous (parental) tumor antigens is delayed and therefore was not detected when both PSMA-expressing and non PSMA-expressing tumor cells were implanted at the same time as was done in FIG. 6C.

To test this hypothesis, it was determined whether immune responses generated against the NMD controlled products as shown in FIG. 10A “spreads” to tumor antigens expressed in parental tumors. As shown in FIGS. 10C (and 10A), T cells isolated 5 days after implantation of SMG-1 shRNA expressing tumor cells, namely tumor cells in which NMD was inhibited, recognized NMD-inhibited, but not parental tumor cells, implying that the immune response was directed against the NMD controlled products which were upregulated upon NMD inhibition but not against endogenous antigens expressed in the parental tumor cells. It was also hypothesized that if induction of immunity against the NMD products leads to epitope spread, at later time points the mice will generate immune responses also against the parental tumor. Indeed, as shown in FIG. 10C when T cell responses were measured 30 days post tumor inoculation an immune response was elicited against parental tumor cells which was comparable in magnitude to that elicited against the NMD controlled products. This experiment, therefore, provides immunological evidence that immune responses against NMD-controlled products “spreads” to endogenous tumor antigens and that it takes time to develop.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

1. A composition for inhibiting nonsense mediated decay (NMD) pathways comprising an aptamer-oligonucleotide molecule wherein said aptamer is specific for a desired target cell and the oligonucleotide molecule inhibits nonsense mediated decay pathways.

2. The composition of claim 1, wherein said oligonucleotide molecule comprising at least one of a short interfering RNA (siRNA); a micro-interfering RNA (miRNA); antisense oligonucleotides; a small, temporal RNA (stRNA); a short, hairpin RNA (shRNA), or combinations thereof.

3. The composition of claim 1, wherein the oligonucleotide molecule inhibits function and/or expression of at least one factor associated with the NMD pathway comprising at least one of: RENT1, RENT2, eIF4A, UPF1, UPF2, UPF3B, RNPS1, Y14, MAGOH, NMD1, SMG, or combinations thereof.

4. The composition of claim 1, wherein said target cell comprising: a tumor cell, an infected cell, a tissue specific cell, an adipocyte, a stem cell, an immune cell, an organ specific cell or a transformed cell.

5. The composition of claim 1, wherein said aptamer-oligonucleotide molecules comprising one or aptamers, wherein each aptamer is specific for at least one target molecule on a desired target cell and/or one or more oligonucleotides specific for at least one desired target molecule.

6. The composition of claim 1, wherein the aptamer-oligonucleotide molecules, further comprising one or more molecules to promote intracellular delivery, cytoplasmic delivery, bioavailability, or combinations thereof.

7. The composition of claim 6, wherein the aptamer-oligonucleotide comprising at least one of: polylysine, polyarginine, Antennapedia-derived peptides, HIV derived tat peptide, a fusogenic peptide from influenza hemagglutinin protein, a 9mer Arg oligopeptide (SEQ ID NO: 30), peptide transporters, intracellular localization domain sequences, or combinations thereof.

8. The composition of claim 6, wherein said molecules promoting bioavailability comprising at least one of: cholesterol, polyethylene glycol, or combinations thereof.

9. The composition of claim 1, wherein the aptamer-oligonucleotide molecule, comprising aptamer molecules having one or more nucleotide substitutions.

10. The composition of claim 9, wherein the nucleotide substitutions comprise at least one of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants, analogs or combinations thereof.

11. The composition of claim 1, wherein the at least one aptamer is linked to each other and/or the at least one oligonucleotide by at least one linker molecule.

12. The composition of claim 11, wherein at least one aptamer is linked to at least one oligonucleotide by at least one linker molecule.

13. The composition of claim 11, wherein said linker molecule comprising: nucleotide, non-nucleotide, or mixed nucleotide/non-nucleotide molecules.

14. The composition of claim 11, wherein the one or more linker molecules comprising about 2 nucleotides length up to about 50 nucleotides in length.

15. The composition of claim 11, wherein the non-nucleotide linker comprises abasic nucleotide, polyether, polyamine, polyamide, peptide, carbohydrate, lipid, polyhydrocarbon, or polymeric compounds having one or more monomeric units.

16. A composition for inducing novel antigens in abnormal cells, comprising: a multi-domain molecule having at least one target specific domain and at least one domain, which modulates expression and function of molecules associated with nonsense mediated decay pathways.

17. The composition of claim 16, wherein the multi-domain molecule comprises at least one target specific domain and at least two domains which modulate expression and function of one or more molecules associated with nonsense mediated decay pathways.

18. The composition of claim 16, wherein the multi-domain molecule comprises at least two target specific domains and at least one domain which modulates expression and function of one or more molecules associated with nonsense mediated decay pathways.

19. The composition of claim 16, wherein the target specific domains comprise specificities for similar target molecules, different target molecules, or combinations thereof.

20. The composition of claim 16, wherein the domains which modulate expression and function of one or more molecules associated with nonsense mediated decay pathways modulate the expression and function of similar targeted molecules, different targeted molecules or combinations thereof.

21. The composition of claim 16, wherein the target specific domains are specific for target cell molecules, the target cell comprising: a tumor cell, an infected cell, a tissue specific cell, an adipocyte, a stem cell, an immune cell, an organ specific cell or a transformed cell.

22. The composition of claim 16, wherein the oligonucleotide molecules comprising at least one of a short interfering RNA (siRNA); a micro-interfering RNA (miRNA); antisense oligonucleotides; a small, temporal RNA (stRNA); a short, hairpin RNA (shRNA), or combinations thereof.

23. The composition of claim 16, wherein the molecules associated with nonsense mediated decay pathways, comprising at least one of: RENT1, RENT2, eIF4A, UPF1, UPF2, UPF3B, RNPS1, Y14, MAGOH, NMD1, SMG, or combinations thereof.

24. A method of inducing or enhancing immunogenicity of a target cell in vitro or in vivo and modulating an antigen specific immune response in patient comprising:
obtaining a composition comprising at least one aptamer conjugated to at least one oligonucleotide molecule wherein the aptamer is specific for a desired target cell and the oligonucleotide is specific for a molecule associated with at least one factor associated with a nonsense mediated decay pathway (NMD);
administering the composition in a therapeutically effective amount to the patient; and,
inducing or enhancing the immunogenicity of a target cell and modulating an antigen specific immune response in patient.

25. The method of claim 24, wherein the oligonucleotide molecule comprising at least one of a short interfering RNA (siRNA); a micro-interfering RNA (miRNA); antisense oligonucleotides; a small, temporal RNA (stRNA); a short, hairpin RNA (shRNA), or combinations thereof.

26. The method of claim 24, wherein the oligonucleotide molecule inhibits at least one factor associated with the NMD pathway comprising at least one of: RENT1, RENT2, UPF1, UPF2, UPF3B, RNPS1, Y14, MAGOH, NMD1 or SMG.

27. The method of claim 24, wherein said target cell comprising: a tumor cell, an infected cell, a tissue specific cell, an adipocyte, a stem cell, an immune cell, an organ specific cell or a transformed cell.

28. The method of claim 24, wherein said composition comprising one or more aptamers, wherein each aptamer is specific for at least one target molecule on a desired target cell.

29. The method of claim 24, wherein an immune cell is optionally co-stimulated comprising administering to a patient co-stimulatory inducing agent is optionally administered to the patient.

30. The method of claim 29, wherein an immune cell co-stimulatory agent targets one or more molecules comprising: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

31. The method of claim 29, wherein immune cells comprise T cells (T lymphocytes), B cells (B lymphocytes), antigen presenting cells, dendritic cells, monocytes, macrophages, myeloid suppressor cells, natural killer (NK) cells, NK T cells, suppressor cells, T regulatory cells (Tregs), cytotoxic T lymphocytes (CTLs), CTL lines, CTL clones, CTLs from tumor, inflammatory, or other infiltrates and subsets thereof.

32. An aptamer-oligonucleotide molecule comprising at least one aptamer specific for a marker of a target cell and at least one interference or antisense oligonucleotide specific for a desired polynucleotide of the target cell.

33. The aptamer-oligonucleotide molecule of claim 32, wherein the oligonucleotide molecule comprising at least one of a short interfering RNA (siRNA); a micro-interfering RNA (miRNA); antisense oligonucleotides; a small, temporal RNA (stRNA); a short, hairpin RNA (shRNA), or combinations thereof.

34. The aptamer-oligonucleotide molecule of claim 32, wherein the oligonucleotide molecule inhibits at least one factor associated with the NMD pathway comprising at least one of: RENT1, RENT2, UPF1, UPF2, UPF3B, RNPS1, Y14, MAGOH, NMD1, SMG, or combinations thereof.

35. The aptamer-oligonucleotide molecule of claim 32, wherein the at least one aptamer is linked to the at least interference or antisense oligonucleotide by at least one linker molecule.

36. The aptamer-oligonucleotide molecule of claim 35, wherein the linker molecule comprises nucleotide, non-nucleotide, or mixed nucleotide/non-nucleotide linker joining the one or more aptamers to one or more interference or antisense oligonucleotide molecules.

37. The aptamer-oligonucleotide molecule of claim 35, wherein the one or more linker molecules comprising about 2 nucleotides length up to about 50 nucleotides in length.

38. The aptamer-oligonucleotide molecule of claim 35, wherein the non-nucleotide linker comprises abasic nucleotide, polyether, polyamine, polyamide, peptide, carbohydrate, lipid, polyhydrocarbon, or polymeric compounds having one or more monomeric units.

39. The aptamer-oligonucleotide molecule of claim 35, wherein the aptamer-interference RNA or aptamer-antisense oligonucleotide fusion molecule comprises one or more nucleotide substitutions.

40. The aptamer-oligonucleotide molecule of claim 39, wherein the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants, analogs, or combinations thereof.

41. The aptamer-oligonucleotide molecule of claim 35, wherein said molecule further comprising one or more moieties promote intracellular delivery, cytoplasmic delivery, bioavailability, or combinations thereof.

42. The aptamer-oligonucleotide molecule of claim 41, wherein the one or more moieties comprising at least one of: polylysine, polyarginine, Antennapedia-derived peptides, HIV derived tat peptide, a fusogenic peptide from influenza hemagglutinin protein, a 9mer Arg oligopeptide (SEQ ID NO: 30), peptide transporters, peptide transduction domains, intracellular localization domain sequences, or combinations thereof.

43. The aptamer-oligonucleotide molecule of claim 41, wherein the moieties promoting bioavailability comprising at least one of: cholesterol, polyethylene glycol, or combinations thereof.

44. A method of up-regulating existing and/or inducing new or novel antigens on a cell's surface comprising:
obtaining a composition comprising at least one aptamer conjugated to at least one oligonucleotide molecule wherein the aptamer is specific for a desired target cell and the oligonucleotide is specific for a molecule associated with at least one factor associated with a nonsense mediated decay pathway (NMD);
administering the composition in a therapeutically effective amount to the patient; and,
up-regulating existing and/or inducing new or novel antigens on a cell's surface.

45. The method of claim 44, wherein the oligonucleotide molecule comprising at least one of a short interfering RNA (siRNA); a micro-interfering RNA (miRNA); antisense oligonucleotides; a small, temporal RNA (stRNA); a short, hairpin RNA (shRNA), or combinations thereof.

46. The method of claim 44, wherein the oligonucleotide molecule inhibits at least one factor associated with the NMD pathway comprising at least one of: RENT1, RENT2, UPF1, UPF2, UPF3B, RNPS1, Y14, MAGOH, NMD1, SMG or combinations thereof.

47. The method of claim 44, wherein said target cell comprising: a tumor cell, an infected cell, a tissue specific cell, an abnormal cell, an adipocyte, a stem cell, an immune cell, an organ specific cell or a transformed cell.

48. The method of claim 44, wherein said composition comprising one or more aptamers, wherein each aptamer is specific for at least one target molecule on a desired target cell.

49. The method of claim 44, wherein increasing existing and/or inducing new or novel antigens expressed by a cell targeted by the aptamer-oligonucleotide compositions induces an immune response.

50. The method of claim 49, wherein the immune response is directed to the target cell.


SEQUENCE LISTING (32 sequences)

SEQ ID NO: 1 - RNA, 71 nt, Artificial Sequence
  Description of Artificial Sequence Synthetic oligonucleotide
gggaggacgaugcggaucagccauguuuacgucacuccuugucaauccucaucggcagacgacucgcccga

SEQ ID NO: 2 - DNA, 32 bp, Artificial Sequence
  Description of Artificial Sequence Synthetic primer
taatacgactcactatagggaggacgatgcgg

SEQ ID NO: 3 - DNA, 37 bp, Artificial Sequence
  Description of Artificial Sequence Synthetic primer
aagcgttatgtttggtggaagtcgggcgagtcgtctg

SEQ ID NO: 4 - DNA, 37 bp, Artificial Sequence
  Description of Artificial Sequence Synthetic primer
aagccatgactaacactgaaatcgggcgagtcgtctg

SEQ ID NO: 5 - DNA, 37 bp, Artificial Sequence
  Description of Artificial Sequence Synthetic primer
aaaattctccgaacgtgtcactcgggcgagtcgtctg

SEQ ID NO: 6 - DNA, 21 bp, Artificial Sequence
  Description of Artificial Sequence Synthetic oligonucleotide
aauucuccgaacgugucactt

SEQ ID NO: 7 - DNA, 21 bp, Artificial Sequence
  Description of Artificial Sequence Synthetic oligonucleotide
gcguuauguuugguggaagtt

SEQ ID NO: 8 - DNA, 21 bp, Artificial Sequence
  Description of Artificial Sequence Synthetic oligonucleotide
gccaugacuaacacugaaatt

SEQ ID NO: 9 - DNA, 38 bp, Artificial Sequence
  Description of Artificial Sequence Synthetic primer
gatcagcggccgcgccaccatgtggaatctccttcacg

SEQ ID NO: 10 - DNA, 47 bp, Artificial Sequence
  Description of Artificial Sequence Synthetic primer
gttaagtcgacgaggatcctcgagaatcctcttaggctacttcactc

SEQ ID NO: 11 - DNA, 36 bp, Artificial Sequence
  Description of Artificial Sequence Synthetic primer
gatcagcggccgctgcagaaggtcgggcaggaagag

SEQ ID NO: 12 - DNA, 31 bp, Artificial Sequence
  Description of Artificial Sequence Synthetic primer
gttaagcatgcccacactggactagtggatc

SEQ ID NO: 13 - DNA, 64 bp, Artificial Sequence
  Description of Artificial Sequence Synthetic oligonucleotide
gatcaattctccgaacgtgtcacttcctgacccaaagtgacacgttcggagaatttttttgtac

SEQ ID NO: 14 - DNA, 54 bp, Artificial Sequence
  Description of Artificial Sequence Synthetic oligonucleotide
aaaaaaattctccgaacgtgtcactgggtcaggaagtgacacgttcggagaatt

SEQ ID NO: 15 - DNA, 65 bp, Artificial Sequence
  Description of Artificial Sequence Synthetic oligonucleotide
gatcgcgttatgtttggtggaagaacctgacccattcttccaccaaacataacgcttttttgtac

SEQ ID NO: 16 - DNA, 57 bp, Artificial Sequence
  Description of Artificial Sequence Synthetic oligonucleotide
aaaaaagcgttatgtttggtggaagaatgggtcaggttcttccaccaaacataacgc

SEQ ID NO: 17 - DNA, 64 bp, Artificial Sequence
  Description of Artificial Sequence Synthetic oligonucleotide
gatcgccaccaaagacatgaggaaacctgacccatttcctcatgtctttggtggctttttgtac

SEQ ID NO: 18 - DNA, 56 bp, Artificial Sequence
  Description of Artificial Sequence Synthetic oligonucleotide
aaaaagccaccaaagacatgaggaaatgggtcaggtttcctcatgtctttggtggc

SEQ ID NO: 19 - protein, 8 aa, Artificial Sequence
  Description of Artificial Sequence Synthetic peptide
SIINFEKL

SEQ ID NO: 20 - DNA, 53 bp, Artificial Sequence
  Description of Artificial Sequence Synthetic primer
ccatggtgagtataataaattttgaaaaacttcacctgactcctgaggagaag

SEQ ID NO: 21 - DNA, 17 bp, Artificial Sequence
  Description of Artificial Sequence Synthetic primer
gggtgttggcgggtgtc

SEQ ID NO: 22 - DNA, 20 bp, Artificial Sequence
  Description of Artificial Sequence Synthetic primer
ccacactgtgcccatctacg

SEQ ID NO: 23 - DNA, 21 bp, Artificial Sequence
  Description of Artificial Sequence Synthetic primer
gatcttcatggtgctaggagc

SEQ ID NO: 24 - DNA, 20 bp, Artificial Sequence
  Description of Artificial Sequence Synthetic primer
gcccatcgtgtttgctttgg

SEQ ID NO: 25 - DNA, 23 bp, Artificial Sequence
  Description of Artificial Sequence Synthetic primer
tctcgttcccagtggtgttacag

SEQ ID NO: 26 - RNA, 19 nt, Artificial Sequence
  Description of Artificial Sequence Synthetic primer
acccggggcuaauguugac

SEQ ID NO: 27 - RNA, 24 nt, Artificial Sequence
  Description of Artificial Sequence Synthetic primer
cuugguaauguuaggcguuuucuc

SEQ ID NO: 28 - DNA, 21 bp, Artificial Sequence
  Description of Artificial Sequence Synthetic primer
accaccgtagaacgcagatcg

SEQ ID NO: 29 - DNA, 19 bp, Artificial Sequence
  Description of Artificial Sequence Synthetic primer
cctgaacttctcaggatcc

SEQ ID NO: 30 - protein, 9 aa, Artificial Sequence
  Description of Artificial Sequence Synthetic peptide
RRRRRRRRR

SEQ ID NO: 31 - RNA, 93 nt, Artificial Sequence
  Description of Artificial Sequence Synthetic oligonucleotide
gggaggacgaugcgcaucagccauguuuacgucacuccuugucaauccucaucggcagacgacucgcccgaauuucaguguuagucauggcuu

SEQ ID NO: 32 - RNA, 93 nt, Artificial Sequence
  Description of Artificial Sequence Synthetic oligonucleotide
gggaggacgaugcggaucagccauguuuacgucacuccuugucaauccucaucggcagacgacucgcccgaacuuccaccaaacauaacgcuu